(19) United States
(12) Patent Application Publication
(10) Pub. No.: US 2026/0225001 A1
(43) Pub. Date: Aug. 6, 2026

(54) COMPOSITIONS AND METHODS FOR PURIFYING BIOLOGICAL FLUIDS

(71) Applicant: North Carolina State University, Raleigh, NC (US)

(72) Inventors: Sobhana Sripada, Raleigh, NC (US); Stefano Menegatti, Raleigh, NC (US)

(21) Appl. No.: 19/152,281

(22) PCT Filed: Jan. 31, 2024
(86) PCT No.: PCT/US2024/013783

§ 371 Date: Jul. 30, 2025

Related U.S. Application Data
(60) Provisional application No. 63/482,412, filed on Jan. 31, 2023.

(51) Int. Cl. B01D 15/38; B01J 20/32; C07K 1/22; C07K 5/103; C07K 5/107; C07K 5/11; C07K 5/113; C07K 17/14

(52) U.S. Cl. CPC B01D 15/3804; B01J 20/3204; B01J 20/3212; C07K 1/22; C07K 5/101; C07K 5/1016; C07K 5/1019; C07K 5/1021; C07K 17/14

(57) ABSTRACT

The present disclosure provides materials and methods related to the purification of a biologic from a biological fluid. In particular, the present disclosure provides compositions, and related methods, comprising peptide ligands capable of removing process-related impurities (e.g., host cell proteins, nucleic acids, and media components) and product-related impurities (e.g., product fragments, product aggregates, and inactive forms derived from product degradation by or association with other species in the cell culture harvest) from biological fluids during the production of a biologic.

### CROSS REFERENCE TO RELATED APPLICATIONS

This application claims priority to and the benefit of U.S. Provisional Patent Application No. 63/482,412 filed Jan. 31, 2023, which is incorporated herein by reference in its entirety and for all purposes.

### SEQUENCE LISTING STATEMENT

The contents of the electronic sequence listing titled (NCSU-41575-601.xml; Size: 7,894 bytes; and Date of Creation: Jan. 31, 2024) is herein incorporated by reference in its entirety.

### FIELD

The present disclosure provides materials and methods related to the purification of a target biologic from a biological fluid. In particular, the present disclosure provides compositions, and related methods, comprising peptide ligands capable of removing process-related impurities and product-related impurities from biological fluids during the process of producing a biologic.

### BACKGROUND

The engineering of cell lines in the Pichia genus has been pursued for more than two decades—by improving the design of plasmid, promoters, and signal peptides—to achieve high productivity and fidelity of high-value therapeutics. In particular, the species Komagataella phaffii (K. phaffii) is currently utilized as an expression host for a variety of proteins including antibodies—whole monoclonals and engineered fragments—as well as fusion proteins, virus-like-particles, etc. These efforts have been conducted in concert with system biology to better understand K. phaffii's genetic repertoire as well as the temporal evolution of transcriptomics and proteomics across the various phases of cell growth under different conditions. Nonetheless, a recent SWOT analysis on the adoption of K. phaffii as a platform for protein production highlighted the need to intensity efforts towards secretion rate and reducing protease-mediated product degradation. While capable of both intra- and extra-cellular protein production, K. phaffii features an innately slow secretory trafficking. These issues can be mitigated by improving gene regulation or developing strains with lower proteases content, although these modifications can negatively impact cell growth rates and viability. Therefore, cell engineering and culture efforts must be adjuvated by purification technologies capable of removing K. phaffii host cell proteins (HCPs) with enzymatic and/or immunogenic activity as early as possible in the manufacturing pipeline.

### SUMMARY

Embodiments of the present disclosure include a composition for purifying a target biologic from a biological fluid. In accordance with these embodiments, the composition includes at least one peptide ligand that is at least four amino acids in length and comprises: (i) at least one charged amino acid, and (ii) at least one amino acid comprising a side chain capable of hydrogen bond formation.

In some embodiments, the at least one charged amino acid is arginine (R), histidine (H), lysine (K), and/or glutamate (E). In some embodiments, the at least one charged amino acid is not aspartate (D).

In some embodiments, the at least one amino acid comprising a side chain capable of hydrogen bond formation is tyrosine (Y), tryptophan (W), and/or glutamine (Q). In some embodiments, the at least one amino acid comprising a side chain capable of hydrogen bond formation is not phenylalanine (F).

In some embodiments, the at least one peptide ligand comprises alanine (A) or valine (V). In some embodiments, the at least one peptide ligand does not comprise isoleucine (I), leucine (L), or proline (P). In some embodiments, an arginine (R) residue is flanked by a tyrosine (Y) residue and/or a tryptophan (W) residue. In some embodiments, a valine (V) residue is flanked by a tyrosine (Y) residue and/or a tryptophan (W) residue. In some embodiments, an alanine (A) residue is flanked by a lysine (K) residue.

In some embodiments, the at least four amino acids are hydrophilic amino acids.

In some embodiments, at least two of the at least four amino acids are hydrophobic amino acids.

In some embodiments, the at least one peptide ligand is selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the at least one peptide ligand comprises at least two peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the at least one peptide ligand comprises at least three peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the at least one peptide ligand comprises at least four peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the at least one peptide ligand comprises at least five peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the at least one peptide ligand comprises at least six peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the at least one peptide ligand comprises at least seven peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the composition comprises YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

In some embodiments, the at least one peptide ligand binds at least one host cell protein (HCP), at least one high-risk HCP, at least one host cell nucleic acid, aggregates of the target biologic, and/or an impurity derived from the target biologic.

In some embodiments, the at least one peptide ligand exhibits a K_{D}<10⁻⁵ M for the HCP, the host cell nucleic acid, the aggregates of the target biologic, and/or the impurity derived from the target biologic.

In some embodiments, the at least one peptide ligand comprises a linker. In some embodiments, the linker is bound to the C-terminus of the peptide ligand, and wherein the linker comprises a Glyₙ or a [Gly-Ser-Gly]ₘ, wherein 6≥n≥1 and 3≥m≥1.

In some embodiments, the at least one peptide ligand is bound to a solid support. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate, polyolefin, polystyrene, polyester, polyether, polysaccharide, iron oxide, silica, titania, zirconia, and/or derivatives thereof.

In some embodiments, the biological fluid comprises a cell culture supernatant and/or a cellular lysate. In some embodiments, the biological fluid is derived from a yeast cell. In some embodiments, the yeast cell is selected from the group consisting of P. pastoris, S. cerevisiae, and S. boulardii, or any derivatives or variants thereof.

In some embodiments, the target biologic is one or more of a protein, peptide or polypeptide; an oligonucleotide or a polynucleotide; a virus or a virus-like particle; an exosome or an extracellular vesicle; a cell or cell organelle; or a small molecule. In some embodiments, the biological fluid comprises a pH from about 3.0 to about 9.0. In some embodiments, the biological fluid comprises a conductivity of about 1 mS/cm to about 50 mS/cm.

In some embodiments, the at least one peptide ligand exhibits a maximum equilibrium binding capacity (Qₘₐₓ) of at least about 5.0 g of host cell proteins per liter of resin.

In some embodiments, the at least one peptide ligand exhibits a dynamic binding capacity (DBC_{10%}) from about 5 g/L resin to about 70 g/L resin.

Embodiments of the present disclosure also include an adsorbent comprising any of the compositions described herein.

Embodiments of the present disclosure also include a method of purifying a target biologic from a biological fluid. In accordance with these embodiments, the method includes contacting a composition comprising any of the peptide ligands described herein, or an adsorbent comprising any of the peptide ligands described herein, with a biological fluid comprising a target biologic; and collecting the effluent in flow-through mode. In some embodiments, the effluent comprises the target biologic, and the at least one peptide ligand binds and retains at least one host cell protein (HCP), at least one high-risk HCP, at least one host cell nucleic acid, aggregates of the target biologic, and/or an impurity derived from the target biologic.

In some embodiments, the method further comprises performing affinity chromatography on the biological fluid comprising the target biologic before or after contacting the composition comprising the at least one peptide ligand of any of claims 1 to 35.

In some embodiments, the method is performed under static binding conditions. In some embodiments, the method is performed under dynamic binding conditions.

In some embodiments, the method results in a yield of the target biologic of at least about 50%.

In some embodiments, the method results in a purity of the target biologic of at least about 75%.

### BRIEF DESCRIPTION OF THE DRAWINGS

FIGS. 1A-1C: (A) Flowchart describing the identification and classification of high-risk K. phaffii host cell proteins (HR-HCPs) based on homology to CHO, immunogenicity designation (predicted by IEDB), and protease activity; (B) Histogram of 15-mer fragments generated per K. phaffii HCP identified in the cell culture feed: the fragments were utilized to score the cognate HCP using the IEDB MHC-II immunogenicity prediction tool; (C) Venn diagram showing the overlaps in the classification of HCPs among the various risk groups, which were ultimately chosen to compile the list of HR-HCPs presented in Table 1.

FIGS. 2A-2D: HCP binding isotherms obtained by incubating PichiaGuard TP-650M resin with solutions of K. phaffii HCPs at concentrations ranging between 0-1 mg/mL in either (A) 20 mM sodium acetate at pH 5.7 or (B) 25 mM sodium phosphate at pH 7.2. Values of cumulative logarithmic removal of K. phaffii HCPs afforded by CaptoAdhere, CaptoQ, CHO LigaGuard, and PichiaGuard TP-650M resins loaded (residence time: 1 minute) with X-33 feedstocks with HCP titer of ~0.4 mg/mL and conditioned in either (C) 20 mM sodium acetate at pH 5.7 or (D) 25 mM sodium phosphate at pH 7.2.

FIGS. 3A-3D: (A) Connected colors plot indicating significant differences in mean HCP abundance among flow-through fractions; groups connected with different colors feature significantly different HCP abundances calculated as an average value for the group; (B) Contour plots of K. phaffii HCP abundance ratios registered in the effluents obtained by loading CaptoQ, LigaGuard, and PichiaGuard resins loaded (residence time: 1 minute) with X-33 feedstocks with HCP titer of ~0.4 mg/mL and conditioned in either 20 mM sodium acetate at pH 5.7 or 25 mM sodium phosphate at pH 7.2: individual HCPs are aligned on the x-axis, while the number of collected flow-through fractions is on the y-axis; red to green indicates high to low ratio of individual HCP in the effluent vs. feedstock as detected by nanoLC-MS; (C) Flowchart describing the designation of removal status for ‘high-risk’ HCP (HR-HCPs); (D) Number of individual HR-HCPs removed by CaptoQ, LigaGuard, and PichiaGuard resins grouped by risk category; the values outside the Venn diagrams indicate the number of HCPs that were not removed.

FIGS. 4A-4C: (A) Total number of HCPs removed from a X-33 K. phaffi feedstock (ScFv13R4 titer of ~1 mg/mL, HCP titer of ~0.4 mg/mL, 20 mM sodium acetate at pH 5.7) by PichiaGuard-TP650 resin operated at the residence time of 1 minute as a function of resin loading (namely, 4, 9, and >14 mg of proteins per mL of resin); the number of removed HCPs was determined via proteomic analysis of the flow-through fractions via nanoLC-MS/MS; (B) Number of individual HR-HCPs removed by PichiaGuard-TP650 resin grouped by risk category; the values outside the Venn diagrams indicate the number of HCPs that were not removed; (C) SDS-PAGE gel (native conditions) of the K. phaffi cell culture harvest containing ScFv13R4 and mAb (lanes 2-4), and the flow-through fractions generated by loading the harvests on PichiaGuard-TP650 resin at the residence time of 1 minute (5-7 for ScFv13R4; 8 and 9 for mAb) and final column wash (lane 8 for scFv13R4 and lane 9 for mAb).

FIGS. 5A-5B: (A) Protocol of Filter-Aided Sample Preparation (FASP) implemented to execute the bottom-up proteomic analysis of K. phaffii CCF and flow-through fractions obtained by loading the CCF on control (CaptoQ, CaptoAdhere, CHO LigaGuard™) and experimental (PichiaGuard-TP50F and PichiaGuard-TP650M) resins; (B) Distribution of physicochemical properties (i.e., molecular weight, isoelectric point (pI), and GRAVY indices) of HCPs identified in K. phaffii CCF (top row) and specifically of their “high-risk” subset (bottom row).

FIGS. 6A-6C: (A) Visualization of Channel 1 (green, positive for HCP-binding) and Channel 2 (red, positive for polyclonal Ab binding) from the microfluidic peptide library screening setup showing select HMBA-ChemMatrix resin beads coupled with test peptides that had high HCP selectivity; (B) Frequency and distribution of amino acids identified in K. phaffii HCP binding peptides and their properties; (C) Desalting chromatograms obtained from gel-permeation chromatography conducted to buffer-exchange K. phaffii cell cultures used in the current study, corresponding to clarification of culture with (right) and without (left) protease-inhibitors.

FIGS. 7A-7E: (A) Leverage plots of ‘significant’ variables generated by the DOE model within the control study group viz., resin, buffer and the interaction term between them; (B) Interaction plot showing trends between independent variables and HCP cLRV observed across performed experiments where A,P represent acetate and phosphate buffers, (i)-(ii)-(iii)-(iv) represent PichiaGuard, CHO LigaGuard, CaptoAdhere and CaptoQ respectively; (C) Prediction profiler showing the desirability of choosing PichiaGuard resin operated with Acetate buffer for achieving a goal of maximizing cLRV; (D) F-statistics of variables tested in the DOE mode that show buffer type and loading influence HCP clearance significantly; (E) fractional log reduction of HCP observed with PichiaGuard CCF treated with and without protease inhibitors.

FIGS. 8A-8C: (A, B) trends of individual high-risk HCPs across different flow-through fractions tested in both reference and PichiaGuard groups respectively, across both acetate and phosphate buffer groups. Within each square, the smaller marker represents groups CHO LigaGuard (top) and PichiaGuard TP-50F (bottom) and the larger markers CaptoQ (top) and PichiaGuard TP-650M (bottom); (C) Total number of bound HCPs identified in each flow-through fraction via nano-LC-MS/MS within each experimental condition performed across resin type and buffer condition.

FIGS. 9A-9C: (A) Yield and purity observed in mAb purification from K. phaffii CFF; (B) SDS-PAGE of high-salt chase fractions post PichiaGuard processing showing a small degree of product binding to the column, possibly due to association with other HCPs at run conditions; (C) Reverse phase chromatograms of individual flow-through fractions obtained from ScFv13R4 purification study compared at different intervals of protein loading (mg protein/mL resin) where changing ratios of impurity peaks (grey) and the product peak (peach) were observed. The ratios reported in this article have been calculated by dividing areas under the curves between these retention times respectively.

FIGS. 10A-10B: PichiaGuard performance on (A) scFv and (B) mAb purification from continuous fermentations of recombinant Pichia pastoris cultures.

FIGS. 11A-111B: (A) Collection of flow-through chromatograms of each peptide sequence within the PichiaGuard ensemble compared against the latter (control, last panel). (B) HCP and IgG binding to each peptide observed as a function of loading.

FIGS. 12A-12G: (A-C) Summary of protein properties calculated for HCPs identified within the load samples. (D-G) Grouping and analysis of various properties for each HCP based on its abundance within the load sample.

FIG. 13: Contour plots of HCP binding per individual peptide sequence forming the PichiaGuard ensemble. High HCP binding based on the number of species bound is indicated in red and lower number in blue. This representation does not provide a view of the total number of HCPs identified in the feed sample and hence is suitable only for an inter-peptide comparison.

FIGS. 14A-14I: (A-H) Comparison of total number of HCPs bound by each peptide versus PichiaGuard. (I) Table comparing peptide properties based on their composition and subsequent coverage of HCP binding provided by them. The last two columns contain normalized values of HCP and IgG binding under non-competitive conditions, which help assess selectivity and capacity of each peptide towards HCP binding.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “sequence identity” generally refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “target” or “target biologic” generally refers to a target protein, peptide, polypeptide, nucleic acid, ribonucleoprotein complex, nucleic acid construct, supramolecular construct, virus, viral construct, virus-like particle, cell, organelle, small molecule, and any combinations thereof, which may be present in a sample (e.g., biological fluid) comprising one or more process-related impurities and/or product-related substances. In some embodiments, the target or target biologic is an antibody or any antigen binding fragment/derivative thereof (e.g., monoclonal or polyclonal antibody).

As used herein, the term “host cell protein” or “HCP” refers to any protein produced or encoded by the organism used to produce a recombinant polypeptide product and unrelated to the intended product. For example, HCPs are generally undesirable in a final drug substance.

As used herein, a “mixture” comprises a target biologic of interest (for which purification is desired) and one or more contaminant or impurity. In some embodiments, the mixture is produced from a host cell or organism that expresses the protein of interest (either naturally or recombinantly). Such mixtures include, for example, cell cultures, cell lysates, and clarified bulk (e.g., clarified cell culture supernatant).

As used herein, a “derivative” with respect to a peptide or polypeptide generally has the amino acid sequence of a reference peptide or variant, but additionally comprises a chemical modification of one or more of its amino acid side groups, α-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine 8-amino groups, N-alkylation of arginine, histidine, or lysine, alkylation of glutamic or aspartic carboxylic acid groups, and deamidation of glutamine or asparagine. Modifications of the terminal amino include, without limitation, the desamino, N-lower alkyl, N-di-lower alkyl, constrained alkyls (e.g., branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily-skilled peptide chemist. The α-carbon of an amino acid may be mono- or dimethylated.

Additionally, as used herein, a “variant” with respect to a peptide or polypeptide generally refers to a peptide or polypeptide whose base amino acid sequence was derived from that of a reference peptide or polypeptide. A variant can include conservative and/or non-conservative amino acid substitutions (including non-natural amino acids and L and D forms).

### 2. Compositions and Methods for Removing Process and Product-Related Impurities

Current biotherapeutic formulations contain a final average of ~20 ng HCP per mg drug product, typically represented by less than ten species. Although K. phaffii naturally produces fewer HCPs than its mammalian counterparts, failure to reduce their titer below a prescribed limit puts at risk the stability of the product and the safety of the patient. Decades of production of therapeutic monoclonal antibodies (mAbs) in Chinese hamster ovary (CHO) cells has pointed at the presence of HCPs that conjugate an inherent toxic or immunogenic activity with the ability to escape purification by coeluting with the mAb product. Recently, research groups have assessed the harmful potential of HCPs secreted by non-mammalian and microbial cell lines employed—of forthcoming—in biopharmaceutical manufacturing. However, the identity and values of safety threshold of K. phaffii HCPs and their ability to evade clearance by current chromatographic technology has been but barely investigated. For example, recent K. phaffii engineering efforts have been associated with increased co-expression of isomerases and proteases whose CHO homologues are known as immunogenic and difficult-to-remove, although their potential interference with downstream processing is as of yet unknown.

Current processes for the purification of K. phaffii-secreted proteins rely on a train of operations including cell separation, multiple chromatographic steps, and final concentration or filtration. As in the CHO pipeline, the chromatographic segment typically comprises an affinity-based capture step followed by polishing via ion exchange and hydrophobic interaction modalities. As these cells are harvested at high densities, a liter of culture fluid may contain up to a gram of HCPs, whose distribution of physicochemical and biochemical properties varies with the target product and culture conditions. Such process- and product-dependent diversity of the K. phaffii secretome, combined with the current dearth of knowledge of its impact on purification technology and—most importantly—the lack of affinity technology dedicated to the removal of HCPs increases the risk of contaminants present in polished pools.

Developing a true platform process, capable of sustaining the diversity in titer and biomolecular make-up of industrial feedstocks, can benefit significantly by introducing an affinity resin with robust HCP and hcDNA capture activity. In prior work, the paradigm of “flow-through affinity chromatography” was explored and an adsorbent was developed—named LigaGuard™-functionalized with peptide ligands designed to target the whole CHO proteome spectrum. LigaGuard™ was demonstrated by purifying therapeutic mAbs from an ensemble of seven CHO harvests: when utilized alone, LigaGuard™ afforded a logarithmic HCPs removal value (HCP LRV) ranging between 1.5-2.5; when coupled with an affinity resin operated in bind-and-elute mode, the two-step process afforded cumulative HCP and hcDNA LRVs~4, corresponding to a final HCP level of 8 ppm. Additionally, LigaGuard™ managed to clear high-risk and hard-to-remove HCPs—such as Cathepsins and Phospholipases—thus demonstrating it potential to de-risk and simplify the chromatographic pipeline of protein purification.

In this study, experiments were conducted to expand the study to yeast expression systems by conducting a bioprocess-targeted proteomic survey of K. phaffii cell culture harvests, proposing a mechanism for identifying and annotating HR-HCPs and by developing an adsorbent—named PichiaGuard—for the removal of K. phaffii HCPs and hcDNA via flow-through affinity chromatography. In the first part, common HCPs were identified and characterized in the K. phaffii secretome and their risk and immunogenicity profiles were evaluated using bioinformatics tools. The resultant biomolecular information was leveraged to design and screen a peptide library against the K. phaffii secretome to identify selective HCP-targeting peptide ligands. The discovered ligands were used to formulate PichiaGuard, which was evaluated in terms of HCP binding capacity and purification for monoclonal full mAb and a ScFv fragment from clarified K. phaffii fluids. A proteomics analysis of the effluents was conducted to evaluate the HCP capture performance of the peptide-based resins. Globally, the results indicated that PichiaGuard outperforms state-of-the-art chromatographic resins in terms of both capture of K. phaffii HCPs—notably, among them, aspartic proteases, peptidases, ribosomal subunit proteins, heat shock proteins etc.—and product recovery, demonstrating its value for the purification of high-value biotherapeutics produced by yeast hosts.

a. Compositions

K. phaffii is a versatile expression system that is increasingly utilized to produce biological therapeutics—including enzymes, engineered antibodies, and gene-editing tools—that feature multiple subunits and complex post-translational modifications. Two major roadblocks limit the adoption of K. phaffii in industrial biomanufacturing: its proteome, while known, has not been linked to downstream process operations and detailed knowledge is missing on problematic host cell proteins (HCPs) that endanger patient safety or product stability; furthermore, the purification toolbox has not evolved beyond the capture of monospecific antibodies, and few solutions are available for engineered antibody fragments and other protein therapeutics. To unlock the potential of yeast-based biopharmaceutical manufacturing, this study presents (i) a secretome survey of K. phaffii cell culture harvests that highlights HCPs with predicted immunogenicity, ability to cause product instability by proteolysis or degradation of excipients, and potential to interfere with purification operations via product association or co-elution; and (ii) a novel affinity adsorbent functionalized with peptide ligands that target the whole spectrum of K. phaffii HCPs—PichiaGuard—designed for the enrichment of therapeutic proteins in flow-through mode. The PichiaGuard adsorbent features high HCP binding capacity (~25 g per liter of resin) and successfully purified a monoclonal antibody and an ScFv fragment from clarified K. phaffii harvests, affording up to 80% product yield, and a >300-fold removal of HCPs. Notably, PichiaGuard outperformed commercial ion exchange and mixed-mode resins in removing high-risk HCPs—including aspartic proteases, ribosomal subunits, and other peptidases—thus demonstrating its value in modern biopharmaceutical processing.

In accordance with this, embodiments of the present disclosure include compositions and methods for purifying a target biologic from a biological fluid. In accordance with these embodiments, the composition includes at least one peptide ligand that is at least four amino acids in length and comprises: (i) at least one charged amino acid, and (ii) at least one amino acid comprising a side chain capable of hydrogen bond formation.

As would be recognized by one of ordinary skill in the art based on the present disclosure, some amino acids are known to be electrically charged. In general, lysine (K), arginine (R) and histidine (H) are known to be amino acids having a positive charge (positively charged amino acids). Aspartic acid (D), glutamic acid (E), and such are known to be amino acids having a negative charge (negatively charged amino acids). In addition, alanine (A), asparagine (N), cysteine (C), glutamine (Q), glycine (G), isoleucine (I), leucine (L), methionine (M), phenylalanine (F), proline (P), serine (S), threonine (T), tryptophan (W), tyrosine (Y), valine (V), and the like are known to be amino acids that do not have a charge, or nonpolar amino acids. In some embodiments, and as described further herein, at least one charged amino acid that is present in the peptide ligands of the present disclosure includes arginine (R), histidine (H), lysine (K), and/or glutamate (E). In some embodiments, at least one charged amino acid that is not present in the peptide ligands of the present disclosure is aspartate (D).

As would be recognized by one of ordinary skill in the art based on the present disclosure, some amino acids comprising side chains that are capable of hydrogen bond formation (e.g., hydrogen bond donor and/or a hydrogen bond acceptor). These amino acids generally include methionine (M), serine (S), threonine (T), asparagine (N), glutamine (Q), cysteine (C), aspartic acid (D), glutamic acid (E), phenylalanine (F), lysine (K), alanine (A), glycine (G), tyrosine (Y), and tryptophan (W). In some embodiments, at least one amino acid comprising a side chain capable of hydrogen bond formation that is present in the peptide ligands of the present disclosure includes tyrosine (Y), tryptophan (W), and/or glutamine (Q). In some embodiments, at least one amino acid comprising a side chain capable of hydrogen bond formation that is not present in the peptide ligands of the present disclosure is phenylalanine (F).

In some embodiments, and as described further herein, at least one peptide ligand comprises alanine (A) or valine (V). In some embodiments, the at least one peptide ligand does not comprise isoleucine (I), leucine (L), or proline (P). In some embodiments, an arginine (R) residue is flanked (i.e., directly adjacent to) by a tyrosine (Y) residue and/or a tryptophan (W) residue. In some embodiments, a valine (V) residue is flanked by a tyrosine (Y) residue and/or a tryptophan (W) residue. In some embodiments, an alanine (A) residue is flanked by a lysine (K) residue.

As would be recognized by one of ordinary skill in the art based on the present disclosure, some amino acids are hydrophilic or polar in nature. Generally, amino acids considered to be hydrophilic or polar amino acids include serine (S), threonine (T), cysteine (C), asparagine (N), glutamine (Q), and tyrosine (Y). In some embodiments, and as described further herein, the peptide ligands of the present disclosure can include one or more hydrophilic or polar amino acids. In some embodiments, two of the at least four amino acids in the peptide ligands of the present disclosure are hydrophilic amino acids. In some embodiments, three of the at least four amino acids in the peptide ligands of the present disclosure are hydrophilic amino acids. In some embodiments, four of the at least four amino acids in the peptide ligands of the present disclosure are hydrophilic amino acids.

As would be recognized by one of ordinary skill in the art based on the present disclosure, amino acids that are considered to be hydrophobic (i.e., have hydrophobic side chains) include glycine (G), alanine (A), valine (V), leucine (L), isoleucine (I), proline (P), phenylalanine (F), methionine (M), and tryptophan (W). In some embodiments, two of the at least four amino acids in the peptide ligands of the present disclosure are hydrophobic amino acids. In some embodiments, three of the at least four amino acids in the peptide ligands of the present disclosure are hydrophobic amino acids. In some embodiments, four of the at least four amino acids in the peptide ligands of the present disclosure are hydrophobic amino acids.

In some embodiments, at least one peptide ligand present in the compositions of the present disclosure used to purify a target biologic from a biological fluid is selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof. In some embodiments, at least one peptide ligand of the present disclosure comprises at least two peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof. In some embodiments, at least one peptide ligand of the present disclosure comprises at least three peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof. In some embodiments, at least one peptide ligand of the present disclosure comprises at least four peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof. In some embodiments, at least one peptide ligand of the present disclosure comprises at least five peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof. In some embodiments, at least one peptide ligand of the present disclosure comprises at least six peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof. In some embodiments, at least one peptide ligand of the present disclosure comprises at least seven peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof. In some embodiments, the composition of the present disclosure comprises YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

As would be understood by one of ordinary skill in the art based on the present disclosure, the compositions and peptide ligands of the present disclosure can be used to purify any biological target from a biological fluid. For example, the target biologic can be a polypeptide, a protein, an oligonucleotide, a polynucleotide, a virus or a viral capsid, a portion of the viral capsid, a cell or a cell organelle, or a small molecule. In some embodiments, the target biologic is a protein, such as an antibody, an antibody fragment, an antibody-drug conjugate, a drug-antibody fragment conjugate, a Fc-fusion protein, a hormone, an anticoagulant, a blood coagulation factor, a growth factor, a morphogenic protein, a therapeutic enzyme, an engineered protein scaffold, an interferon, an interleukin, or a cytokine. As would be understood by one of ordinary skill in the art based on the present disclosure, the target biologic can be any protein, peptide, or polypeptide produced in a cell, including any endogenous, exogenous, or recombinant proteins produced by a cell, and the methods and compositions described herein can facilitate their purification from HCPs. In some embodiments, at least one peptide ligand of the present disclosure binds at least one HCP, at least one high-risk HCP, at least one host cell nucleic acid, aggregates of the target biologic, and/or an impurity derived from the target biologic.

In some embodiments, the target biologic can be a virus, viral capsid, or viral vector propagated in a cell. In some embodiments, such viruses, viral capsids, or viral vectors are engineered to deliver genetic material into cells for gene therapy, oncolytic applications, or vaccination; therefore, the various embodiments of the present disclosure can be used to purify the target biologic viruses, viral capsids, or viral vectors before they are administered to a cell or a subject. For example, the target biologic can be a retrovirus (RV), an adenovirus (AV), an adeno-associated virus (AAV), a lentivirus (LV), a baculovirus, or a herpes simplex virus (HSV). As would be understood by one of ordinary skill in the art based on the present disclosure, the target biologic can be any viral vector produced in a cell, and the methods and compositions described herein can facilitate their purification from HCPs.

In some embodiments, the target biologic can be a cell in a stem cell, a progenitor cell, or an immune effector cell. In some embodiments, the immune effector cell includes, but is not limited to, a T cell or a Natural Killer (NK) cell, including immune effector cells engineered to include a chimeric antigen receptor (CAR), such as CAR-T cells and CAR-NK cells. In some embodiments, the target biologic can be an extracellular vesicle or an exosome.

In accordance with these embodiments, the compositions of the present disclosure can be used in the production of any biologic, including but not limited to, biologic molecules such as antibodies and antibody fragments (e.g., single-chain variable fragments (scFv), single-chain antibodies (scAb), and fragment antigen binding molecules (Fab fragments), diabodies, glycoengineered antibodies, bi-specific antibodies, antibody-drug conjugates, as well as any combinations, derivatives, variants, and fusions thereof. For example, the peptide compositions of the present disclosure can be used to purify any of the currently available therapeutic antibodies, including but not limited to, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ustekinumab (Stelara), infliximab, and bevacizumab. In some embodiments, the present disclosure includes a composition comprising any of the peptide ligands disclosed herein, and one of the above-recited therapeutic antibodies.

Product- and/or process-related impurities or contaminants can include any protein, peptide, polypeptide, and/or nucleic acid that is not desirable in a purified composition comprising a target biologic. For example, product- and/or process-related impurities can include any fragments or aggregates of the target biologic that are not desired in a purified composition. In some embodiments, the product- and/or process-related impurities can include an intact target biologic that has undergone a chemical or biochemical modification (e.g., enzyme modification of the amino acid sequence of the target biologic or its profile of post-translational modifications), or an intact target biologic that has become associated with an impurity (e.g., and has been rendered inactive).

In accordance with these embodiments, at least one peptide ligand of the present disclosure binds at least one HCP, at least one host cell nucleic acid, aggregates of the target biologic, and/or an impurity derived from the target biologic. The one or more HCPs can be any host cell protein which one would want to remove from a mixture and is independently selected from the proteome of the host cell expressing the one or more target biologics. Examples of host cell proteins include, but are not limited to, those listed in Table 1. In some embodiments, a peptide ligand of the present disclosure binds a specific HCP, or a specific type or class of HCPs, such that the peptide ligand can be used with the compositions and methods described herein to remove that specific HCP, or specific type or class of HCPs, from a biological fluid to facilitate the purification of a target biologic.

In some embodiments, the peptide ligands of the present disclosure can be conjugated to a linker. In some embodiments, the linker can facilitate display of a peptide ligand onto a solid support, which allows for better capture of an HCP, for example. In other embodiments, the peptide ligands provided herein are not conjugated to a linker, but can still be bind to HCPs and be removed from a cell culture fluid through other means. In some embodiments, the one or more peptide ligands comprise a linker on the C-terminus of the peptide. The C-terminus linker comprise a linker according to the following structure: Glyₙ or a [Gly-Ser-Gly]ₘ, wherein 6≥n≥1 and 3≥m≥1. The C-terminus linker can be any suitable linker including, but not limited to GSG and GGG.

In some embodiments, at least one peptide ligand of the present disclosure exhibits a K_{D} that is at least about 10⁻³ M for the HCP, the host cell nucleic acid, and/or the target biologic aggregate. In some embodiments, at least one peptide ligand of the present disclosure exhibits a K_{D} that is at least about 10⁻⁴ M for the HCP, the host cell nucleic acid, and/or the target biologic aggregate. In some embodiments, at least one peptide ligand of the present disclosure exhibits a K_{D} that is at least about 10⁻⁵ M for the HCP, the host cell nucleic acid, and/or the target biologic aggregate. In some embodiments, at least one peptide ligand of the present disclosure exhibits a K_{D} that is at least about 10⁻⁶ M for the HCP, the host cell nucleic acid, and/or the target biologic aggregate. In some embodiments, at least one peptide ligand of the present disclosure exhibits a K_{D} that is at least about 10⁻⁷ M for the HCP, the host cell nucleic acid, and/or the target biologic aggregate. In some embodiments, at least one peptide ligand of the present disclosure exhibits a K_{D} that is at least about 10⁻⁸ M for the HCP, the host cell nucleic acid, and/or the target biologic aggregate. In some embodiments, at least one peptide ligand of the present disclosure exhibits a K_{D} that is at least about 10⁻⁹ M for the HCP, the host cell nucleic acid, and/or the target biologic aggregate. In some embodiments, at least one peptide ligand of the present disclosure exhibits a K_{D} that is at least about 10⁻¹⁰ M for the HCP, the host cell nucleic acid, and/or the target biologic aggregate.

In some embodiments, at least one peptide ligand of the present disclosure exhibits a K_{D} from about 10⁻¹⁰ M to about 10⁻³ M for the HCP, the host cell nucleic acid, and/or the target biologic aggregate. In some embodiments, at least one peptide ligand of the present disclosure exhibits a K_{D} from about 10⁻⁹ M to about 10⁻³ M for the HCP, the host cell nucleic acid, and/or the target biologic aggregate. In some embodiments, at least one peptide ligand of the present disclosure exhibits a K_{D} from about 10⁻⁸ M to about 10⁻³ M for the HCP, the host cell nucleic acid, and/or the target biologic aggregate. In some embodiments, at least one peptide ligand of the present disclosure exhibits a K_{D} from about 10⁻⁷ M to about 10⁻³ M for the HCP, the host cell nucleic acid, and/or the target biologic aggregate. In some embodiments, at least one peptide ligand of the present disclosure exhibits a K_{D} from about 10⁻⁶ M to about 10⁻³ M for the HCP, the host cell nucleic acid, and/or the target biologic aggregate. In some embodiments, at least one peptide ligand of the present disclosure exhibits a K_{D} from about 10⁻⁵ M to about 10⁻³ M for the HCP, the host cell nucleic acid, and/or the target biologic aggregate. In some embodiments, at least one peptide ligand of the present disclosure exhibits a K_{D} from about 10⁻⁴ M to about 10⁻³ M for the HCP, the host cell nucleic acid, and/or the target biologic aggregate. In some embodiments, at least one peptide ligand of the present disclosure exhibits a K_{D} that is lower than about 10⁻⁵ M for the HCP, the host cell nucleic acid, and/or the target biologic aggregate. In some embodiments, at least one peptide ligand of the present disclosure exhibits a K_{D} from about 10⁻¹⁰ M to about 10⁻⁴ M for the HCP, the host cell nucleic acid, and/or the target biologic aggregate. In some embodiments, at least one peptide ligand of the present disclosure exhibits a K_{D} from about 10⁻⁹ M to about 10⁻³ M for the HCP, the host cell nucleic acid, and/or the target biologic aggregate. In some embodiments, at least one peptide ligand of the present disclosure exhibits a K_{D} from about 10⁻⁹ M to about 10⁻⁴ M for the HCP, the host cell nucleic acid, and/or the target biologic aggregate. In some embodiments, at least one peptide ligand of the present disclosure exhibits a K_{D} from about 10⁻⁹ M to about 10⁻⁵ M for the HCP, the host cell nucleic acid, and/or the target biologic aggregate. In some embodiments, at least one peptide ligand of the present disclosure exhibits a K_{D} from about 10⁻⁸ M to about 10⁻³ M for the HCP, the host cell nucleic acid, and/or the target biologic aggregate. In some embodiments, at least one peptide ligand of the present disclosure exhibits a K_{D} from about 10⁻⁸ M to about 10⁻⁴ M for the HCP, the host cell nucleic acid, and/or the target biologic aggregate. In some embodiments, at least one peptide ligand of the present disclosure exhibits a K_{D} from about 10⁻⁸ M to about 10⁻⁵ M for the HCP, the host cell nucleic acid, and/or the target biologic aggregate. In some embodiments, at least one peptide ligand of the present disclosure exhibits a K_{D} from about 10⁻⁷ M to about 10⁻³ M for the HCP, the host cell nucleic acid, and/or the target biologic aggregate. In some embodiments, at least one peptide ligand of the present disclosure exhibits a K_{D} from about 10⁻⁷ M to about 10⁻⁴ M for the HCP, the host cell nucleic acid, and/or the target biologic aggregate. In some embodiments, at least one peptide ligand of the present disclosure exhibits a K_{D} from about 10⁻⁷ M to about 10⁻⁵ M for the HCP, the host cell nucleic acid, and/or the target biologic aggregate. In some embodiments, at least one peptide ligand of the present disclosure exhibits a K_{D} from about 10⁻⁶ M to about 10⁻³ M for the HCP, the host cell nucleic acid, and/or the target biologic aggregate. In some embodiments, at least one peptide ligand of the present disclosure exhibits a K_{D} from about 10⁻⁶ M to about 10⁻⁴ M for the HCP, the host cell nucleic acid, and/or the target biologic aggregate. In some embodiments, at least one peptide ligand of the present disclosure exhibits a K_{D} from about 10⁻⁶ M to about 10⁻⁵ M for the HCP, the host cell nucleic acid, and/or the target biologic aggregate.

In some embodiments, the peptide ligands of the present disclosure can be linked together to form a larger polypeptide. In other embodiments, the peptide ligands of the present disclosure can be linked to other peptides or polypeptides to form a larger polypeptide. In some embodiments, the at least one peptide ligand is no more than 20 amino acids in length. In some embodiments, the at least one peptide ligand is no more than 16 amino acids in length. In some embodiments, the at least one peptide ligand is no more than 12 amino acids in length. In some embodiments, the at least one peptide ligand is no more than 8 amino acids in length. In some embodiments, the at least one peptide ligand is no more than 4 amino acids in length. In some embodiments, the at least one peptide ligand is from about 4 to about 20 amino acids in length. In some embodiments, the at least one peptide ligand is from about 4 to about 16 amino acids in length. In some embodiments, the at least one peptide ligand is from about 4 to about 12 amino acids in length. In some embodiments, the at least one peptide ligand is from about 4 to about 8 amino acids in length. In some embodiments, the at least one peptide ligand is from about 8 to about 16 amino acids in length. In some embodiments, the at least one peptide ligand is from about 12 to about 20 amino acids in length.

In some embodiments, and as described further herein, the peptide ligands of the present disclosure can be used to purify a target biologic from a biological fluid. In some embodiments, the biological fluid is a cell culture fluid that comprises a cell culture supernatant and/or a cellular lysate. In some embodiments, the biological fluid is derived from a yeast cell. In some embodiments, the yeast cell is selected from the group consisting of P. pastoris, S. cerevisiae, and S. boulardii, or any derivatives or variants thereof.

In some embodiments, the cell culture fluid comprises a pH from about 3.0 to about 9.0. In some embodiments, the cell culture fluid comprises a pH from about 4.0 to about 9.0. In some embodiments, the cell culture fluid comprises a pH from about 5.0 to about 9.0. In some embodiments, the cell culture fluid comprises a pH from about 6.0 to about 9.0. In some embodiments, the cell culture fluid comprises a pH from about 7.0 to about 9.0. In some embodiments, the cell culture fluid comprises a pH from about 3.0 to about 8.0. In some embodiments, the cell culture fluid comprises a pH from about 3.0 to about 7.0. In some embodiments, the cell culture fluid comprises a pH from about 3.0 to about 6.0. In some embodiments, the cell culture fluid comprises a pH from about 4.0 to about 8.0. In some embodiments, the cell culture fluid comprises a pH from about 5.0 to about 7.0.

In some embodiments, the cell culture fluid comprises a conductivity of about 1 to about 50 mS/cm. In some embodiments, the cell culture fluid comprises a conductivity of about 5 to about 50 mS/cm. In some embodiments, the cell culture fluid comprises a conductivity of about 10 to about 50 mS/cm. In some embodiments, the cell culture fluid comprises a conductivity of about 15 to about 50 mS/cm. In some embodiments, the cell culture fluid comprises a conductivity of about 20 to about 50 mS/cm. In some embodiments, the cell culture fluid comprises a conductivity of about 30 to about 50 mS/cm. In some embodiments, the cell culture fluid comprises a conductivity of about 40 to about 50 mS/cm. In some embodiments, the cell culture fluid comprises a conductivity of about 1 to about 40 mS/cm. In some embodiments, the cell culture fluid comprises a conductivity of about 1 to about 30 mS/cm. In some embodiments, the cell culture fluid comprises a conductivity of about 1 to about 20 mS/cm. In some embodiments, the cell culture fluid comprises a conductivity of about 1 to about 15 mS/cm. In some embodiments, the cell culture fluid comprises a conductivity of about 10 to about 40 mS/cm. In some embodiments, the cell culture fluid comprises a conductivity of about 20 to about 30 mS/cm.

In some embodiments, the at least one peptide ligand exhibits a maximum equilibrium binding capacity (Qₘₐₓ) of at least about 5.0 g of host cell proteins per liter of resin. In some embodiments, the at least one peptide ligand exhibits a maximum equilibrium binding capacity (Qₘₐₓ) of at least about 5.5 g of host cell proteins per liter of resin. In some embodiments, the at least one peptide ligand exhibits a maximum equilibrium binding capacity (Qₘₐₓ) of at least about 6.0 g of host cell proteins per liter of resin. In some embodiments, the at least one peptide ligand exhibits a maximum equilibrium binding capacity (Qₘₐₓ) of at least about 6.5 g of host cell proteins per liter of resin. In some embodiments, the at least one peptide ligand exhibits a maximum equilibrium binding capacity (Qₘₐₓ) of at least about 7.0 g of host cell proteins per liter of resin. In some embodiments, the at least one peptide ligand exhibits a maximum equilibrium binding capacity (Qₘₐₓ) of at least about 7.5 g of host cell proteins per liter of resin. In some embodiments, the at least one peptide ligand exhibits a maximum equilibrium binding capacity (Qₘₐₓ) of at least about 8.0 g of host cell proteins per liter of resin. In some embodiments, the at least one peptide ligand exhibits a maximum equilibrium binding capacity (Qₘₐₓ) of at least about 8.5 g of host cell proteins per liter of resin. In some embodiments, the at least one peptide ligand exhibits a maximum equilibrium binding capacity (Qₘₐₓ) of at least about 9.0 g of host cell proteins per liter of resin. In some embodiments, the at least one peptide ligand exhibits a maximum equilibrium binding capacity (Qₘₐₓ) of at least about 9.5 g of host cell proteins per liter of resin. In some embodiments, the at least one peptide ligand exhibits a maximum equilibrium binding capacity (Qₘₐₓ) of at least about 10.0 g of host cell proteins per liter of resin.

In some embodiments, the at least one peptide ligand exhibits a dynamic binding capacity (DBC_{10%}) from about 5 g/L resin to about 70 g/L resin. In some embodiments, the at least one peptide ligand exhibits a dynamic binding capacity (DBC_{10%}) from about 10 g/L resin to about 70 g/L resin. In some embodiments, the at least one peptide ligand exhibits a dynamic binding capacity (DBC_{10%}) from about 20 g/L resin to about 70 g/L resin. In some embodiments, the at least one peptide ligand exhibits a dynamic binding capacity (DBC_{10%}) from about 30 g/L resin to about 70 g/L resin. In some embodiments, the at least one peptide ligand exhibits a dynamic binding capacity (DBC_{10%}) from about 40 g/L resin to about 70 g/L resin. In some embodiments, the at least one peptide ligand exhibits a dynamic binding capacity (DBC_{10%}) from about 50 g/L resin to about 70 g/L resin. In some embodiments, the at least one peptide ligand exhibits a dynamic binding capacity (DBC_{10%}) from about 60 g/L resin to about 70 g/L resin. In some embodiments, the at least one peptide ligand exhibits a dynamic binding capacity (DBC_{10%}) from about 5 g/L resin to about 60 g/L resin. In some embodiments, the at least one peptide ligand exhibits a dynamic binding capacity (DBC_{10%}) from about 5 g/L resin to about 50 g/L resin. In some embodiments, the at least one peptide ligand exhibits a dynamic binding capacity (DBC_{10%}) from about 5 g/L resin to about 40 g/L resin. In some embodiments, the at least one peptide ligand exhibits a dynamic binding capacity (DBC_{10%}) from about 5 g/L resin to about 30 g/L resin. In some embodiments, the at least one peptide ligand exhibits a dynamic binding capacity (DBC_{10%}) from about 5 g/L resin to about 20 g/L resin. In some embodiments, the at least one peptide ligand exhibits a dynamic binding capacity (DBC_{10%}) from about 5 g/L resin to about 10 g/L resin. In some embodiments, the at least one peptide ligand exhibits a dynamic binding capacity (DBC_{10%}) from about 10 g/L resin to about 60 g/L resin. In some embodiments, the at least one peptide ligand exhibits a dynamic binding capacity (DBC_{10%}) from about 25 g/L resin to about 50 g/L resin. In some embodiments, the at least one peptide ligand exhibits a dynamic binding capacity (DBC_{10%}) from about 30 g/L resin to about 60 g/L resin. In some embodiments, the at least one peptide ligand exhibits a dynamic binding capacity (DBC_{10%}) from about 25 g/L resin to about 45 g/L resin.

b. Adsorbents

Further described herein are adsorbents comprising a composition as described above, where each peptide ligand of the composition is conjugated to a support. Supports may comprise, but are not limited to, particles, beads, plastic surfaces, resins, fibers, and/or membranes. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate, polyolefin, polystyrene, polyester, polyether, polysaccharide, iron oxide, silica, titania, zirconia, and/or derivatives thereof. In some embodiments, supports may include microparticles and/or nanoparticles. Each support may be made out of any suitable material including, but not limited to, synthetic or natural polymers, metals, and metal oxides. Some supports may be magnetic, such as a magnetic bead, microparticle and/or nanoparticle. Suitable synthetic polymers include, but are not limited to, polymethacrylate, polyethersulfone, and polyethyleneglycole. Suitable natural polymers include, but are not limited to, cellulose, agarose, and chitosan. Suitable metal oxides include, but are not limited to, iron oxide, silica, titania, and zirconia. Further described herein are adsorbents comprising a composition as described above conjugated to a support.

In some embodiments, the adsorbent comprises a single type of support made from a single type of support material, where all of the peptides in the composition are conjugated to supports formed of the single type of support material. In these embodiments, the composition may comprise one or more different types of peptides, each conjugated to the single type of support made from the single type of support material. In other embodiments, the adsorbent comprises a plurality of types of support. Each type of support may be made of the same type of support material or different types of support materials. In these embodiments, the composition may comprise one or more different types of peptides, each conjugated to a different type of support. In still other embodiments, the peptides of the composition can be conjugated to a soluble compound, for example stimuli-responsive polymer chains to remove HCPs by affinity precipitation.

c. Methods

As described further herein, the present disclosure also provides improved methods for purifying a target biologic from a biological fluid comprising and one or more product- and/or process-related impurities or contaminants, as compared to currently used methods. In some embodiments, the method includes contacting a composition comprising any of the peptide ligands described herein (or any of the adsorbents comprising any of the peptide ligands described herein) with a biological fluid (e.g., a cell culture fluid or cell lysate), and collecting the effluent in flow-through mode, with the effluent comprising the target biologic. In some embodiments, the at least one peptide ligand binds and retains a host cell protein (HCP), a host cell nucleic acid, and/or an aggregate of the target biologic in a retentate.

Further described herein are methods for removing one or more host cell proteins from a mixture comprising the one or more host cell proteins and one or more target biologics. The methods comprise contacting the mixture with a composition or adsorbent described herein. In one embodiment, the contacting between the composition or adsorbent and the mixture results in the binding of the one or more host cell proteins to the composition or adsorbent. In this embodiment, the one or more host cell proteins has a higher binding affinity for the composition, as compared to the one or more target biologics. This results in the preferred binding of the composition to the one or more host cell proteins as compared to the one or more target molecules.

The methods of the present disclosure can further comprise washing the composition or adsorbent to remove one or more unbound target biologics into a supernatant or mobile phase; and then collecting the supernatant or mobile phase containing the one or more unbound target biologics. In an embodiment, the washing step can also occur after the contacting step and after the collection of the supernatant or mobile phase.

In some embodiments, the method can be performed under any binding conditions suitable for use with the composition or adsorbent, including both static binding conditions and dynamic binding conditions. In some embodiments the unbound target biologics are collected into a supernatant when the methods are performed under static binding conditions. In some embodiments the unbound target biologics are collected into a mobile phase when the methods are performed under dynamic binding conditions. The methods of the present disclosure can optionally include flow-through chromatography and weak partition chromatography.

The binding affinity of the compositions and/or adsorbent for the host cell proteins, as compared to the one or more target molecules, can be altered by changes in the following: properties and concentration of the one or more target proteins; the properties and concentration of the host cell proteins; the composition, concentration, and pH of the mixture; and/or the loading conditions and residence time of the contacting and washing steps. Any of these variables can be changed to variables which are suitable according to the methods of the present disclosure and result in increased or decreased binding affinity as required for the present disclosure.

In some embodiments, the contacting step comprises a high ionic strength binding buffer or low ionic strength binding buffer. A low ionic strength binding buffer comprises a buffer of between 1-50 mM NaCl. In one embodiment the low ionic strength binding buffer comprises 20 mM NaCl. A high ionic strength binding buffer comprises a buffer of between 100-500 mM NaCl. In one embodiment the low ionic strength binding buffer comprises 150 mM NaCl.

In some embodiments, the contacting step can comprise a low pH buffer of between pH 5-9. In some embodiments, the contacting step can comprise a low pH buffer of between pH 5-8. In some embodiments, the contacting step can comprise a low pH buffer of between pH 5-7. In some embodiments, the contacting step can comprise a low pH buffer of between pH 6-9. In some embodiments, the contacting step can comprise a low pH buffer of between pH 6-8. In some embodiments, the contacting step can comprise a low pH buffer of between pH 6-7. In some embodiments, the contacting step can comprise a low pH buffer of between pH 7-9. In some embodiments, the contacting step can comprise a low pH buffer of between pH 7-8.

As would be appreciated by one of ordinary skill in the art based on the present disclosure, the methods described herein can be used before or after any purification method typically used to purify and/or isolate a given target molecule. For example, the methods disclosed herein can be used before or after ion exchange chromatography (e.g., cation exchange chromatography, anion exchange chromatography, and/or mixed mode chromatography), before or after affinity chromatography (e.g., Protein A affinity chromatography), and/or before or after size exclusion chromatography or other filtration treatment. In some embodiments, the methods disclosed herein are used after a cell culture fluid has been clarified, but prior to performing a chromatography step (e.g., Protein A affinity chromatography).

In some embodiments, the methods of the present disclosure are particularly suitable for use in the manufacturing of therapeutic antibodies, which can greatly benefit from the adoption of the compositions of peptides and adsorbents of the present disclosure owing to their potential of transforming the downstream process from a pipeline of “batch” chromatographic steps operated in “bind-and-elute” mode to a pipeline of continuous and connected chromatographic train operated in flow-through” mode. However, the methods described herein are also applicable to the purification of other target biologics, such as gene therapy products. These include, for example, viruses for in vivo (e.g., adenovirus and adeno-associated virus) and in vitro (e.g., lentivirus and baculovirus) gene therapy. Unlike proteins, viruses are much larger in size (>20 nm), yet much lower in titer (10¹¹-10¹³ vg/mL, corresponding to g/mL levels, much lower than the typical mg/mL titer of proteins in cell culture harvests) and often in biochemical stability (e.g., all viruses quickly lose infectivity when exposed to the typical elution conditions (low pH) currently utilized for their purification; specific adeno-associated virus serotypes are very prone to irreversible adhesion and aggregation; lentiviruses are very sensitive to pH variations outside of the physiological range). As a result, affinity-based purification in capture-and-elute mode fails to provide the product yield and quality requested by clinics and biotech companies worldwide. The compositions and methods of the present disclosure circumvent these issues by enabling flow-through purification of viruses. The key benefits of this approach include, but are not limited to, (i) flowing the cell culture fluid from the bioreactor to capture HCPs while excluding the viruses by size (upon adjusting the pore diameter, the HCPs can enter the pores, while the viruses are excluded), thereby improving product recovery; (ii) rapid clearance of HCPs at minimal residence time (upon adjusting the particle diameter), thereby improving product stability; (iii) operating in flow-through mode avoids virus adsorption on the resin and exposure to variations in conductivity and pH (associated to washing/elution buffers in current bind-and-elute affinity purification), thereby reducing product aggregation and preserving its transduction activity.

Additional applications of the compositions and methods of the present disclosure include the detection of low-abundance proteins in biological fluids, such as cell culture harvests, plant/tissue extracts, bodily fluids (e.g., blood, serum, plasma, sweat, urine, saliva). In this context, the prevalence of mass spectrometry (MS)-based analytical techniques for process monitoring and diagnostic applications has posed an accent on the need to enrich and/or isolate low-abundance proteins that are often key markers of product quality of disease. MS-based analysis relies on the ionization of the analyte species in the sample: abundant analytes, due to their higher titer, capture most of the electrons, at the expense of low-titer analytes, which become undetected. The compositions and methods of the present disclosure can overcome these limitations by concentrating the HCPs and releasing them in a controlled fashion: (i) all HCPs are initially captured on the adsorbent; (ii) the HCPs are “eluted” using a linear or a step-wise gradient, which progressively releases cohorts of HCPs from the adsorbent and directly into the analytical equipment. The low-abundance proteins are present in the eluted stream at a much higher concentration and are more likely to be detected.

In accordance with these embodiments, the compositions and methods of the present disclosure can be used in the production of any biologic, including but not limited to, biologic molecules such as antibodies (monoclonal and polyclonal) and antibody fragments (e.g., single-chain variable fragments (scFv), single-chain antibodies (scAb), fragment antigen binding molecules (Fab fragments), diabodies, glycoengineered antibodies, bi-specific antibodies, antibody-drug conjugates, as well as any combinations, derivatives, variants, and fusions thereof. For example, the peptide compositions and methods of the present disclosure can be used to purify any of the currently available therapeutic antibodies, including but not limited to, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ustekinumab (Stelara), infliximab, and bevacizumab. In some embodiments, the present disclosure includes a method for purifying any of the above-recited therapeutic antibodies by combining a cell culture fluid comprising one of the above-recited antibodies with a composition comprising any of the peptide ligands disclosed herein.

### 3. Examples

The success of K. phaffii in pharmaceutical biomanufacturing is scripted in its rapid growth in chemically defined media, low susceptibility to virus contamination, facile expression inducibility, and ability to secrete human proteins with correct post-translational modifications at high titer and purity. These upstream-related benefits call for convergent efforts in the downstream toolbox, especially in the context of continuous processing and isolation of products for which dedicated affinity adsorbents are not available. In this context, new chromatographic tools are needed that provide orthogonality in removing process-related impurities and/or complementary to commercial adsorbents in clearing residual impurities to ensure the safety of patients treated with complex biological drugs. This work has contributed to this field by introducing the paradigm of flow-through affinity chromatography in the form of ‘Guard’ resins, whose HCP and hcDNA capture activity provides an orthogonal step to affinity resins for product capture or a complementary step to IEX and MM resins for product polishing. In this context, the heuristic power of designed peptide libraries coupled with high-throughput dual-fluorescence screening was leveraged to identify peptide ligands that capture persistent and high-risk contaminants; furthermore, advanced analytics, such as proteomics, were integrated to demonstrate the potential of these ligands in biomanufacturing. The initial efforts, conducted in the domain of full mAb and CHO HCPs, were supported by an established portfolio of analytical technologies and a wealth of literature highlighting target HCPs.

In the present disclosure, the ‘Guard’ technology was extended to K. phaffii HCPs by introducing PichiaGuard, the first affinity adsorbent dedicated to the clearance of process-related impurities present in Pichia cell culture harvests. While the selection of K. phaffii HCP-binding peptides was streamlined by a robust ligand identification technology, the evaluation of the resulting PichiaGuard adsorbent faced challenges related to the quantification of product recovery and contaminant removal. In particular, the dearth of relevant literature on the toxicological, immunological, bioprocess-relevant aspects of Pichia HCPs compelled us to formulate criteria for identifying species that pose—or are expected to pose—a risk to patient's health (e.g., immunogenicity via B-cell activation) or product stability, and can compromise the efficiency of the purification process (e.g., co-elution with the product or persistence throughout the downstream train). Having established an analytical panel, experimental results demonstrated that PichiaGuard affords up to 99% reduction of HCPs and hcDNA from a native K. phaffii cell culture supernatant, when challenged with a harvest containing a therapeutic product—a full mAb or an scFv fragment—thus providing product enrichment just by just impurity removal. Most importantly, PichiaGuard's ability to clear immunogenic (e.g., 40S ribosomal proteins, Histone H3, Thioredoxin peroxidase, Phosphatidylinositol transfer protein, Translation initiation factor eIF4G etc.), proteolytically (e.g., Lon protease, Aspartic proteases—vacuolar, GPI-anchored and plasma membrane-attached forms) and enzymatically active (e.g., Lysophospholipase, Catalase, Peptidase A1, Ydj1p cooperating heat shock protein etc.) high-risk HCPs is key to process robustness and product safety. These results warrant a path forward for PichiaGuard, chiefly its integration with commercial IEX and MM resins in a straight-through process.

The accompanying Examples are offered as illustrative as a partial scope and particular embodiments of the disclosure and are not meant to be limiting of the scope of the disclosure.

### Example 1

Proteomics analysis and immunogenicity annotation of the K. phaffii secretome. The remarkable progresses in mass spectrometry and statistical correlation models achieved in the last decade have transformed the proteomics of biological fluids from a complex endeavor to a routine procedure. The proteomic analysis of bioreactor harvests and bioprocess fractions has enabled the compilation of databases that highlight features of host cell proteins (HCPs) that are relevant in biopharmaceutical manufacturing, such as the ability to associate with a therapeutic product, escape clearance by specific chromatographic modalities, enzymatic activity that causes product degradation or destabilization, and immunogenicity or toxicity. To date, the attention of both academic and industrial communities has focused on the proteomics of Chinese hamster ovary (i.e., Crisetulus griseus) cells, given their widespread used in the production of monoclonal antibodies (mAbs), as exemplified by the databases CHOPPI and CHOGenome.

The recent growth in use of yeast expression systems in biomanufacturing has highlighted the need of producing analogous databases for Pichia hosts. Upon completing the sequencing of the full K. phaffii genome, -omics approaches have indeed been implemented to acquire deeper understanding of the K. phaffii expression system. Proteomic studies conducted by various teams have contributed to establishing public data repositories—chiefly, the Pichia genome—and have highlighted variations in the expression and secretion of HCPs upon changes in feeding strategies (glucose vs. methanol), oxygen levels during cell growth, feed composition, and location of expression (intra- vs. extra-cellular). To date, up to 575 unique proteins have been identified in K. phaffii, with its secretome consisting of at least 335 confirmed species. These databases, however, do not contain annotation on the risk profile of K. phaffii HCPs in the context of bioprocessing.

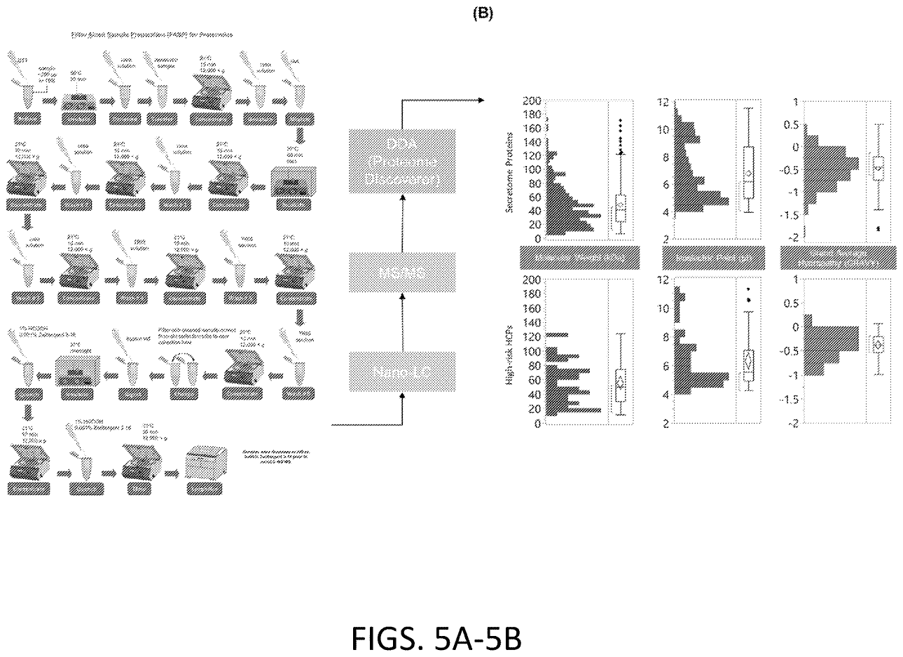

To overcome this gap, a database was built that included K. phaffii HCPs with (i) reported or predicted immunogenicity, (ii) homology with CHO HCPs that have been shown to co-elute with mAbs from different chromatographic modalities, and (iii) enzymatic activity that can compromise the function, structure, and stability of recombinant products. To this end, a nanoLC-MS/MS workflow established in prior work was implemented (FIG. 5A) to conduct a bottom-up proteomic analysis of a supernatant produced by culturing methylotropic wild-type (X-33) K. phaffii and identify HCPs that are secreted along with those released by lysed cells. It was noted that bottom-up proteomics involves proteolytic (tryptic) digestion of bioprocess samples and protein identification via reconstruction of primary sequences at the data-dependent analysis (DDA) stage in nanoLC-MS/MS. While this may lead to the overestimation of certain functional HCPs from pre-existing peptide fragments of HCPs cleaved in the cell culture, it also serves as a challenging scenario for evaluation of novel purification technologies. The resultant spectral data was searched against UniProt K. phaffii database (Proteome ID: UP000000314), which encompasses 5,073 proteins identified across three different Pichia species. The list of identified proteins and their UniProt IDs/RefSeq accession numbers have been determined (data not shown but can be made available upon request). Data interrogation indicated the presence of multiple proteases, with an abundance of aspartic proteases that are especially active in acidic environments (i.e., pH 3-5), which is also a range frequently utilized for product elution at the affinity-based capture step. Indeed, lower product yields caused by proteolytic degradation have often been observed with K. phaffii, whose protease-rich secretome also results in a myriad of HCP fragments that can maintain their immunogenic potential while also being challenging to remove due to non-covalent association to the product. These proteins were identified and tagged ‘risky’ for the analyses.

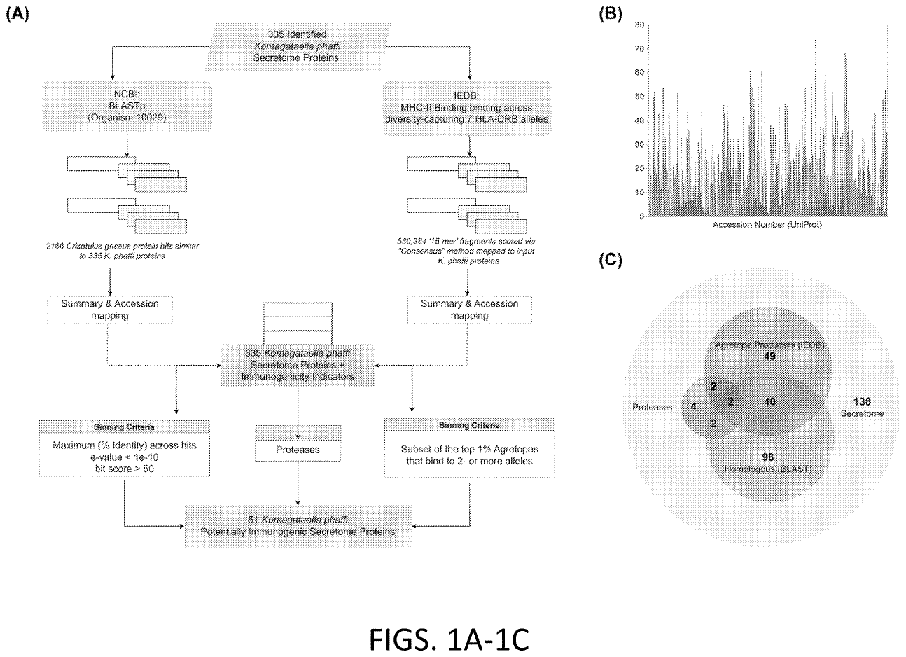

In addition, an extended list of problematic K. phaffii HCPs was compiled by combining reports found in the literature with the estimated homology between K. phaffii HCPs and CHO HCPs with known immunogenicity and a sequence-based ‘Immunogenicity Potential’ score predicted by the NIAID's Immune Epitope Database. A list of 335 proteins identified in the clarified harvest (secretome) by Proteome Discoverer, consistent with previously reported literature, were processed using the workflow outlined in FIG. 1A: the amino acid sequences of the K. phaffii HCPs identified via nanoLC-MS/MS were aligned with a non-redundant database of Cricetulus griseus proteins (NCBI Tax ID: 10029) using the NCBI BLASTp online tool by implementing a stringent definition of “homologous” hit, namely >30% sequence identity, an e-value <10⁻¹⁰, and a bit score >50 based on the Pearson criteria. BLASTp returned 2,166 hits including multiple ‘similar’ sequence hits per each K. phaffii HCP. To avoid increasing redundancy, the maximum number of hits per sequence was capped at 10 followed by application of the discussed criteria. This list was then consolidated by ‘summarizing’ the sequence homology properties using the number of BLAST hits per K. phaffii HCP as a metric, along with the properties mentioned above. In a data sampling of homologous hits with >50% sequence match, K. phaffii counterparts of peptidyl-prolyl isomerases, protein disulfide isomerases and histones H2A/H3/H4, 40S/60S ribosomal proteins etc. have been identified—which have been associated with product aggregation/drug stability in CHO systems.

In parallel, the immunogenicity of K. phaffii HCPs was predicted using the MHC-II binding predictor tool of IEDB by implementing a ‘Consensus Approach’ that estimates the propensity of a protein fragment to promiscuously bind MHC-II alleles. The sequences of K. phaffii HCPs were flanked as 15-mers and tested for binding against 7-alleles (3 loci of HLA-DRB1, 2 loci of DRB3, 1 locus each of DRB4 and DRB5), which have been observed to bind to antigenic peptides, leading to an immune response in 50% general population (note: maximum allelic diversity has been observed with HLA-DRB1, whose 3 variants contribute to capture the diversity within this test set). The consensus approach for scoring MHC-II binding combined the predictions from several algorithms (NN-align, SMM-align, Stumiolo/CombLib, NetMHCIIpan) to ensure broad coverage and low rate of false negativity. The scored 15-mer fragments (n=580,384, (FIG. 1B)) generated in this process were mapped back to the original K. phaffii HCP primary sequences among which a subset of ‘Agretope’—forming HCPs (i.e., consensus percentile rank 10%, per IEDB recommendations) were identified and flagged as ‘risky’ based on the ability of their fragments to bind more than 2 tested allelic variants. As an example, thioredoxin peroxidase and peroxiredoxin-1 both belong to the Peroxiredoxin family of enzymes—the latter of which has been reported as immunogenic in the CHO proteome, and the former has been flagged by the workflow as immunogenic in K. phaffii.

A combination of ‘risky’ proteases identified in the culture harvest were therefore utilized, K. phaffii HCPs with reported or predicted immunogenicity and homology to immunogenic CHO HCPs to identify a set of 51 high-risk HCPs that have a potential for either immunogenicity or product degradation (FIG. 1C and Table 1). Information regarding the HR-HCPs and their selection criteria (IEDB scores, Homology parameters) has been collated (data not included but can be made available upon request). This ensemble of HR-HCPs is non-exhaustive and is meant to serve as a guidance list for scientists developing therapeutic bioprocesses using K. phaffii hosts. This database was used to track the persistence of bioprocess-relevant HCPs throughout the purification pipeline and compare the performance of commercial vs. bespoke chromatographic adsorbents in terms of both global HCP reduction and clearance of high-risk species through rest of the study.

TABLE 1
List of K. phaffii host cell proteins flagged as “high-risk” (HR-HCPs) and their homology to known CHO
HR-HCPs. The high-risk classification includes: (1) HCPs frequently encountered as problematic in bioprocessing,
(2) HCPs with evidence for immunogenic potential, and (3) enzymatic proteases that can cause product degradation.

| UniProt | | | |
|---|---|---|---|
| Accession | | Homologous | Gene Ontology: |
| # | Name | CHO HR-HCP | Molecular Function |
| C4QV16 | Protein component of the large (60S) | | structural constituent of ribosome |
| | ribosomal subunit, nearly identical to | | [GO: 0003735] |
| | Rpl4Ap | | |
| C4QV80 | Dihydrolipoyllysine-residue | | dihydrolipoyllysine-residue |
| | succinyltransferase | | succinyltransferase activity [GO: 0004149] |
| C4QV89 | Heat shock protein that cooperates with | | ATP binding [GO: 0005524]; ATP hydrolysis |
| | Ydj1p (Hsp40) and Ssa1p (Hsp70) | | activity [GO: 0016887] |
| C4QV95 | Ubiquitin-specific protease that | | thiol-dependent deubiquitinase |
| | deubiquitinates ubiquitin-protein | | [GO: 0004843] |
| | moieties | | |
| C4QVY8 | Translation initiation factor eIF4G, | | translation initiation |
| | subunit of the mRNA cap-binding protein | | factor activity |
| | complex (EIF4F) | | [GO: 0003743] |
| C4QY07 | Phosphoglycerate kinase | Phosphoglycerate | ATP binding [GO: 0005524]; |
| | | Kinase 1 | phosphoglycerate kinase activity |
| | | | [GO: 0004618] |
| C4QY27 | Subunit of the SWI/SNF chromatin | | |
| | remodeling complex | | |
| C4QZL4 | Protein component of the large (60S) | 60S Ribosomal | RNA binding [GO: 0003723]; structural |
| | ribosomal subunit | Subunit Protein | constituent of ribosome [GO: 0003735] |
| C4QZQ5 | Elongation factor 2 | | GTP binding [GO: 0005525]; GTPase activity |
| | | | [GO: 0003924] |
| C4QZS3 | Endoplasmic reticulum chaperone BiP | ER Chaperone BiP | ATP binding [GO: 0005524]; ATP hydrolysis |
| | | Precursor | activity [GO: 0016887] |
| C4QZZ6 | Dolichyl-phosphate-mannose--protein | | dolichyl-phosphate-mannose-protein |
| | mannosyltransferase | | mannosyltransferase activity [GO: 0004169] |
| C4R080 | Proteasome endopeptidase complex | Alpha Enolase | proteasomal ubiquitin-independent |
| | | | protein catabolic process [GO: 0010499]; |
| | | | ubiquitin-dependent protein |
| | | | catabolic process |
| | | | [GO: 0006511] |
| C4R080 | Alpha 6 subunit of the 20S proteasome | proteasome | proteolysis involved |
| | | subunit alpha | in protein catabolic |
| | | type 7 isoform x1 | process [GO: 0051603] |
| C4R095 | Kex2 proprotein convertase | | serine-type endopeptidase activity |
| | | | [GO: 0004252]; serine-type |
| | | | endopeptidase inhibitor |
| | | | activity [GO: 0004867] |
| C4R0N8 | Uncharacterized protein GN =PAS_chr2- | | polyubiquitin modification-dependent |
| | 1_0892 PE = 4 SV = 1 | | protein binding [GO: 0031593] |
| C4R0P1 | Glyceraldehyde-3-phosphate | Glyceraldehyde-3 | Enables oxidoreductase activity |
| | dehydrogenase, isozyme 3 | Phosphate | [GO: 0016620]; NAD/NADP binding |
| | | Dehydrogenase | [GO: 0050661] |
| C4R0Q2 | GTP-binding nuclear protein | | GTP binding [GO: 0005525]; GTPase activity |
| | | | [GO: 0003924] |
| C4R0Y3 | Translationally-controlled tumor protein | | cytoskeleton protein involved in |
| | homolog | | translational activity [GO: 0006412] |
| C4R142 | Isocitrate dehydrogenase [NADP] | | isocitrate dehydrogenase (NADP+) activity |
| | | | [GO: 0004450]; magnesium ion binding |
| | | | [GO: 0000287]; NAD binding [GO: 0051287] |
| C4R146 | Protein component of the small (40S) | | structural constituent of ribosome |
| | ribosomal subunit | | [GO: 0003735] |
| C4R1P7 | Acetyl-coenzyme A synthetase | Glutaredoxin-2 | acetate-CoA ligase activity [GO: 0003987]; |
| | | | AMP binding [GO: 0016208]; ATP binding |
| | | | [GO: 0005524] |
| C4R1P9 | Pyruvate Kinase | Pyruvate Kinase | Nucleotide binding [GO: 0000166] |
| | | PKM isoform X1 | and transferase activity |
| | | | [GO: 0016740] |
| C4R2G3 | FK506-binding protein | Peptidyl-prolyl | peptidyl-prolyl |
| | | cis-trans | cis-trans |
| | | isomerase | isomerase activity |
| | | | [GO: 0003755] |
| C4R2H3 | Thioredoxin peroxidase | | thioredoxin peroxidase activity |
| | | | [GO: 0008379]; unfolded protein |
| | | | binding [GO: 0051082] |
| C4R2J7 | Histone H3 | | DNA binding [GO: 0003677]; protein |
| | | | heterodimerization activity [GO: 0046982] |
| C4R2S1 | Catalase | | catalase activity [GO: 0004096]; heme |
| | | | binding [GO: 0020037]; metal ion |
| | | | binding [GO: 0046872] |
| C4R306 | Phosphomannomutase | | phosphomannomutase activity |
| | | | [GO: 0004615] |
| C4R3H8 | Enolase I, a phosphopyruvate hydratase | Beta-enolase | Enables lyase activity [GO: 0016829], |
| | that catalyzes the conversion of 2- | isoform x3 | enables phosphopyruvate hydratase |
| | phosphoglycerate to phospho | | activity [GO: 0004634] |
| C4R3Q7 | Peptidase A1 domain-containing protein | | aspartic-type endopeptidase activity |
| | | | [GO: 0004190] |
| C4R3X8 | ATPase involved in protein folding and | Heat shock | ATP binding [GO: 0005524]; ATP hydrolysis |
| | the response to stress | cognate protein | activity [GO: 0016887] |
| C4R451 | Aa_trans domain-containing protein | Vacuolar | membrane protein [GO: 0005774] |
| | | transporter | |
| C4R458 | Aspartic protease, attached to the | | aspartic-type endopeptidase activity |
| | plasma membrane via a | | [GO: 0004190] |
| | glycosylphosphatidylinositol (GPI) anchor | | |
| C4R493 | Subunit of the core complex of | | translation initiation factor activity |
| | translation initiation factor 3(EIF3) | | [GO: 0003743] |
| C4R4F5 | Putative GPI-anchored aspartic protease | | aspartic-type endopeptidase activity |
| | | | [GO: 0004190] |
| C4R4V6 | Component of the RSC chromatin | | zinc ion binding [GO: 0008270] |
| | remodeling complex | | |
| C4R566 | Phosphatidylinositol/phosphatidylcholine | | phosphatidylcholine transporter activity |
| | transfer protein | | [GO: 0008525]; phosphatidylinositol |
| | | | transfer activity [GO: 0008526] |
| C4R5P4 | Tetrameric phosphoglycerate mutase | Phosphoglycerate | enables catalytic activity [GO: 0004619] |
| | | mutase 1 | |
| C4R5U7 | S-adenosylmethionine synthase | | ATP binding [GO: 0005524]; metal ion |
| | | | binding [GO: 0046872]; methionine |
| | | | adenosyltransferase activity [GO: 0004478] |
| C4R626 | Triosephosphate isomerase | | triose-phosphate isomerase activity |
| | | | [GO: 0004807] |
| C4R6G4 | Non-ATPase base subunit of the 19S | | ubiquitin-dependent protein catabolic |
| | regulatory particle (RP) of the 26S | | process [GO: 0006511] |
| | proteasome | | |
| C4R6G8 | Vacuolar aspartyl protease (Proteinase A) | Cathepsin E | aspartic-type endopeptidase activity |
| | | | [GO: 0004190] |
| C4R6P3 | Protein component of the large (60S) | | structural constituent of ribosome |
| | ribosomal subunit | | [GO: 0003735] |
| C4R703 | Lysophospholipase | Lysosomal | lysophospholipase activity [GO: 0004622]; |
| | | Phospholipase A2 | phosphatidyl phospholipase B activity |
| | | (LPLA2) | [GO: 0102545] |
| C4R7A1 | 40S ribosomal protein S4 | | rRNA binding [GO: 0019843]; structural |
| | | | constituent of ribosome [GO: 0003735] |
| C4R7W9 | Lon protease homolog, mitochondrial | | ATP binding [GO: 0005524]; ATP hydrolysis |
| | | | activity [GO: 0016887]; ATP-dependent |
| | | | peptidase activity [GO: 0004176]; serine- |
| | | | type endopeptidase activity [GO: 0004252] |
| C4R7Y4 | Protein component of the small (40S) | | structural constituent of ribosome |
| | ribosomal subunit | | [GO: 0003735] |
| C4R887 | ATPase involved in protein folding and | | ATP binding [GO: 0005524]; ATP hydrolysis |
| | nuclear localization signal (NLS)-directed | | activity [GO: 0016887] |
| | nuclear transport | | |
| C4R8B8 | Aspartic protease, attached to the | | aspartic-type endopeptidase activity |
| | plasma membrane via a | | [GO: 0004190] |
| | glycosylphosphatidylinositol (GPI) anchor | | |
| | GN = PAS_chr4_0584 PE = 3 SV = 1 | | |
| C4R8R1 | Cytoplasmic thioredoxin isoenzyme of | Periredoxin-1 | protein disulfide reductase activity |
| | the thioredoxin system | family | [GO: 0015035] |
| C4R8U6 | Uncharacterized protein | | GTPase regulator activity [GO: 0030695]; |
| | GN = PAS_chr4_0989 PE = 4 SV = 1 | | metal ion binding [GO: 0046872] |
| C4R938 | Protein disulfide isomerase, | Protein Disulfide | Protein disulfide isomerase activity |
| | multifunctional protein resident in the | Isomerase | [GO: 0003756] |
| | endoplasmic reticulum lumen | | |

| UniProt | | | Effects on |
|---|---|---|---|
| Accession | | K. phaffii HR | Downstream |
| # | Gene Name | Classification | Operations |
| C4QV16 | PAS_chr1-3_0034 | 1, 2 | Recurrent observation in |
| | | | downstream processes |
| C4QV80 | PAS_chr1-3_0094 | 1, 2 | |
| C4QV89 | PAS_chr1-3_0102 | 1, 2, 3 | Sorting and degradation of |
| | | | proteins |
| C4QV95 | PAS_chr1-3_0108 | 1 | Recurrent observation in |
| | | | downstream processes |
| C4QVY8 | PAS_chr1-1_0053 | 1, 2 | |
| C4QY07 | PAS_chr1-4_0292 | 1, 2 | Recurrent observation in |
| | | | downstream processes |
| C4QY27 | PAS_chr1-4_0309 | 1, 2 | |
| C4QZL4 | PAS_chr2-1_0087 | 1, 2 | Recurrent observation in |
| | | | downstream processes |
| C4QZQ5 | PAS_chr2-1_0812 | 1, 2 | Recurrent observation in |
| | | | downstream processes |
| C4QZS3 | PAS_chr2-1_0140 | 1, 2 | Impacts drug quality by |
| | | | increasing aggregation |
| | | | propensity |
| C4QZZ6 | PAS_chr2-1_0212 | 1, 2 | |
| C4R080 | PAS_chr2-1_0291 | 2 | Leads to drug product |
| | | | modification via catalysis |
| | | | of dehydration reaction |
| | | | that converts glycerate to |
| | | | pyruvate |
| C4R080 | PAS_chr2-1_0291 | 2 | Recurrent observation in |
| | | | downstream processes |
| C4R095 | PAS_chr2-1_0304 | 3 | Signal peptidase for |
| | | | secretion, often |
| | | | overexpressed with |
| | | | recombinant gene |
| C4R0N8 | PAS_chr2-1_0892 | 1, 2 | |
| C4R0P1 | PAS_chr2-1_0437 | 1 | Recurrent observation in |
| | | | downstream processes |
| C4R0Q2 | PAS_chr2-1_0449 | 1, 2 | Recurrent observation in |
| | | | downstream processes |
| C4R0Y3 | PAS_chr2-1_0522 | 1, 2 | |
| C4R142 | PAS_chr2-1_0580 | 2 | |
| C4R146 | PAS_chr2-1_0584 | 2 | |
| C4R1P7 | PAS_chr2-1_0767 | 1, 2 | Recurrent observation in |
| | | | downstream processes |
| C4R1P9 | PAS_chr2-1_0769 | 1, 2 | May lead to |
| | | | immunogenicity due to |
| | | | glycolytic action |
| C4R2G3 | PAS_chr2-2_0476 | 1, 2 | Increases aggregation |
| | | | propensity by influencing |
| | | | folding and protein |
| | | | assembly in ER |
| C4R2H3 | PAS_chr2-2_0220 | 1, 2 | |
| C4R2J7 | PAS_c034_0036 | 1, 2 | Recurrent observation in |
| | PAS_chr2-2 0199 | | downstream processes |
| C4R2S1 | PAS_chr2-2_0131 | 1, 2 | Recurrent observation in |
| | | | downstream processes |
| C4R306 | PAS_chr2-2_0053 | 2 | |
| C4R3H8 | PAS_chr3_0082 | 2 | Can lead to drug product |
| | | | modification via catalysis |
| | | | of dehydration reaction |
| | | | that converts glycerate to |
| | | | pyruvate |
| C4R3Q7 | PAS_chr3_1157 | 2, 3 | Aspartic protease |
| C4R3X8 | PAS_chr3_0230 | 1, 2 | Recurrent observation in |
| | | | downstream processes |
| C4R451 | PAS_chr3_0295 | 1, 2 | |
| C4R458 | PAS_chr3_0303 | 2, 3 | Aspartic protease |
| C4R493 | PAS_chr3_0340 | 1, 2 | |
| C4R4F5 | PAS_chr3_0394 | 1, 3 | Aspartic protease |
| C4R4V6 | PAS_chr3_0544 | 1, 2 | |
| C4R566 | PAS_chr3_0655 | 1, 2 | |
| C4R5P4 | PAS_chr3_0826 | 2 | Recurrent observation in |
| | | | downstream processes |
| C4R5U7 | PAS_chr3_0876 | 1, 2 | |
| C4R626 | PAS_chr3_0951 | 1, 2 | Recurrent observation in |
| | | | downstream processes |
| C4R6G4 | PAS_chr3_1083 | 1, 2, 3 | |
| C4R6G8 | PAS_chr3_1087 | 2, 3 | |
| C4R6P3 | PAS_chr4_0041 | 2 | Recurrent observation in |
| | | | downstream processes |
| C4R703 | PAS_chr4_0153 | 2, 3 | Leads to polysorbate |
| | | | degradation by cleaving |
| | | | acyl ester bonds of |
| | | | glycerophospholipids |
| C4R7A1 | PAS_chr4_0246 | 1, 2 | Recurrent observation in |
| | | | downstream processes |
| C4R7W9 | PIM1 | 1, 2, 3 | Recurrent observation in |
| | PAS_chr4_0441 | | downstream processes |
| C4R7Y4 | PAS_chr4_0456 | 1, 2 | Recurrent observation in |
| | | | downstream processes |
| C4R887 | PAS_chr4_0552 | 1, 2 | |
| C4R8B8 | PAS_chr4_0584 | 1, 2, 3 | Aspartic protease |
| C4R8R1 | PAS_chr4_0725 | 2 | |
| C4R8U6 | PAS_chr4_0989 | 1, 2 | |
| C4R938 | PAS_chr4_0844 | 2 | Affects drug product |
| | | | quality by reduction of |
| | | | protein disulfide bonds |
| | | | between cysteine residues |

### Example 2

Identification of K. phaffii HCP-targeting peptide ligands. The sequence-based bioinformatic analysis of the K. phaffii HCPs identified in the harvest fluid presents a rather unique landscape of physicochemical properties (FIG. 5B; note: all values are calculated based on the amino acid sequence of the HCPs): (i) the molecular weight distribution, centered at around 46.14 kDa, is significantly sharper (standard deviation, σ~29.03 kDa) and a narrower (7-193 kDa) compared to its CHO counterpart (σ~35.91, 5-776 kDa); (ii) the values of isoelectric point feature a distinct bimodal distribution peaking at the values of pH 5 and 9.5, with 40% of the species being anionic and 60% cationic at the physiological culture pH, which represents a second sharp difference compared to CHO HCPs (610% anionic and 39% cationic); (iii) the values of grand average hydropathy (GRAVY), varying between −1.84 and 0.49 (σ~0.37), indicate that K. phaffii HCPs are more hydrophilic than CHO HCPs (−1.90 to 0.97; (σ~0.26); and (iv) an average polarity of 50.1% across all K. phaffii HCPs, with most species between a range of 42-62%, indicate a strong propensity to form hydrogen-bond networks.

This analysis informed the design of a library whence peptides could be selected that target the whole spectrum of the K. phaffii secretome: firstly, both positively and negatively charged amino acids (Glu, Lys, His, Arg; Asp was excluded from the library due to the abundance of aspartyl proteases in K. phaffii harvests) were included to address anionic and cationic ensembles of HCPs; furthermore, given the abundance of polar species, neutral amino acids that are hydrogen bond-forming (Gln and the spacer Ser) were included to ensure the formation of hydrogen-bonding interactions; for the same reason, only one aliphatic amino acid (Val; Ile and Leu were excluded) and two aromatic amino acids capable of forming hydrogen-bonds (Tyr and Trp; Phe was excluded) were included; finally, two amino acid linkers, the flexible Gly and the semi-rigid Ala (the rigid Pro was excluded) to sample the broadest possible range of conformations. The multi-polar composition of the library was inspired by that of the ligands forming the CHO LigaGuard™, among which multi-polar peptides hold a prominent role as key contributors to the capture of high-risk and persistent CHO HCPs. A short peptide length of 7 total residues in the format X₁-X₂-X₃-X₄-G-S-G, wherein the 4 residues on the N-terminus form the HCP-binding segment and are varied within the library, while the Gly-Ser-Gly tripeptide on the C-terminus acts as a flexible spacer to enhance ligand display. This format was inspired by prior studies, where it was observed that most of the energy of protein:peptide binding is contributed by the 4 residues on the N-terminus, and the recommendation of using PichiaGuard as a disposable—and ideally single-use—adsorbent, which requires moderate cost and thus short, easy-to-synthesize peptides.

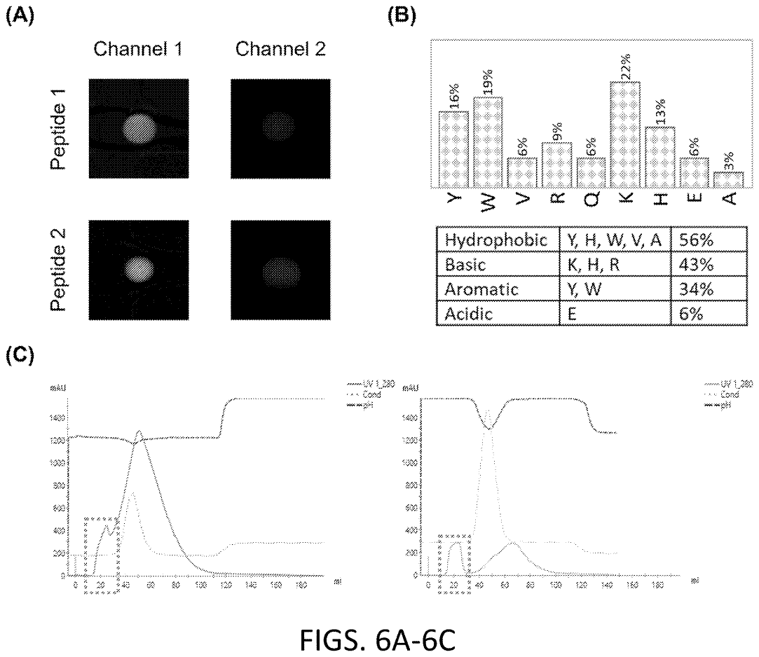

These design criteria were implemented in the synthesis of a One-Bead-One-Peptide (OBOP) combinatorial library on ChemMatrix™ resin, whose translucent, porous, hydrophilic beads are ideal for library selection in competitive mode. To ensure the identification of selective HCP-targeting ligands capable of purifying both full mAbs and engineered mAb-derived products in flow-through mode, an automated orthogonal fluorescence screening was implemented using a bead-imaging-and-sorting device developed in prior work. The workflow utilizes an automated bead-selection algorithm that processes images of the beads in real time, thus enabling the sampling of a large portion of the library and utilizes multiple fluorescence emission wavelengths to select leads with high target-binding strength and selectivity. To that end, a library screening feedstock mimicking industrial harvests was prepared by combining K. phaffii HCPs and human IgG—including whole antibody, Fc/Fab fragments, and nanobody fragments—at a titer of 0.2 mg/mL and 2 mg/mL respectively. The HCPs and IgG species were collectively labeled with a red and a green-fluorescent dye, respectively. The library beads displaying high-intensity red-only fluorescent emission (i.e., strong HCP-only capture behavior at thermodynamic equilibrium) were selected and the peptides carried thereupon were sequenced via Edman degradation, and their sequences were analyzed to identify position-based homology of residues (FIG. 6A) and amino acid frequency in the identified peptides (FIG. 6B).

The sequences present a strong enrichment in aromatic and cationic/H-bonding residues, and a depletion in anionic and aliphatic residues. The physicochemical properties of the candidate ligands are complementary to those of K. phaffii HCPs, thus providing confidence in the outcome of library screening. It was noted that, while the enrichment of cationic residues was anticipated, given the abundance of anionic HCPs, the presence of a population of cationic HCPs (FIG. 5B) seemed to warrant some enrichment of Asp, which was not registered. Accordingly, to ensure the broadest possible HCP-binding activity, eight peptides were selected—namely, RYWV, QEKK, VWHH, EWAK, RYWK, YHKH, RWYQ, WYKK—that feature a diverse amino acid arrangement and composition and are thus expected to encompass a broad spectrum of binding modalities.

### Example 3

Capture of K. phaffii HCPs via flow-through affinity chromatography using PichiaGuard. The selected peptides were conjugated on Toyopearl beads, a polymethacrylate-based chromatographic resin whose mechanical rigidity, particle size (65 μm), and pore diameter (100 nm) are ideal for protein purification via flow-through affinity chromatography. The K. phaffii X-33 cell culture fluid (CCF) utilized to evaluate the HCP binding activity of PichiaGuard was initially diafiltered to (i) remove residual glycerol and methanol as well as fragments resulting from proteolytic activity and residual media components, which are likely to interfere with the protein:peptide interaction (FIG. 6C); (ii) adjust the HCP titer to different concentrations, ranging from 0.01 to 1.5 mg/mL to study the effect of protein concentration on binding kinetics and capacity; and (iii) adjust the buffer composition to either 20 mM sodium acetate at pH 5.7 (2.6 mS/cm) or 20 mM sodium phosphate at pH 7.1 (7 mS/cm) to study the effect of ionic strength and pH on binding kinetics and capacity. These buffers were adopted owing to their kosmotropic character that ensures a native, folded protein state, while promoting affinity interactions; furthermore, the pH chosen to prepare the acetate buffer matches that of K. phaffii culture conditions, while the neutral pH of the phosphate buffer matches that utilized for the CHO HCP-targeting LigaGuard™ and provides a necessary control to interpret the HCP capture results.

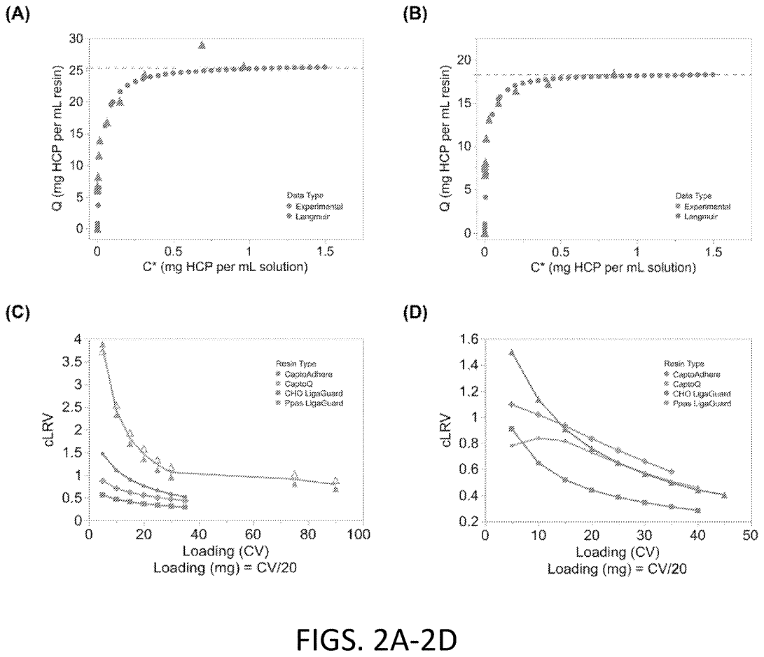

Static binding studies were conducted by incubating the PichiaGuard resin with the conditioned harvests and the residual HCP titer in solution were measured to calculate the equilibrium binding. The resulting adsorption points were fit against Langmuir isotherm curves (FIGS. 2A-2B), from which the values of maximum binding capacity (Qₘₐₓ) and dissociation constant (K_{D}) for the peptide mixture were derived. The rather notable difference in Qₘₐₓ, 25.4 and 18.3 mg HCP per mL resin respectively registered in acetate and phosphate buffers, could be attributed to the different ionic strength of the buffers, wherein the higher conductivity of phosphate buffer shields Coulomb interactions; however, the observation that cationic peptide ligands provide lower binding at pH 7, where the anionic HCPs should be more accentuated, suggests that the electrostatic component of binding is one, but not the dominant, component of the HCP:peptide interaction. Thus, the variation in Qₘₐₓ can also be imputed to the different position in the Hofmeister series of the anions, wherein the less kosmotropic acetate ions promote the formation of ligand-accessible cavities/pockets on HCPs (i.e., salting in) that provide ideal target sites for hydrophobic or aromatic and polar residues in the PichiaGuard peptides to respectively form hydrophobic or π-π and hydrogen bond interactions; at the low concentration in the respective buffers, in fact, the acetate and phosphate ions are not expected to perturb hydrogen bonds and salt bridges between HCPs and peptides. The combination of these effects results in a strong HCP-binding activity by the peptide-functionalized resin, and ultimately a high binding capacity.

The values of K_{D}— 17.5 μM and 30 μM measured in acetate and phosphate buffer, respectively—seem to indicate a moderate HCP:peptide affinity. These values, however, result from treating the K. phaffii HCPs as a single species, a characteristic trait of the evaluation of a chromatographic technology against fluids featuring varying HCP titers and profiles, and commonly found in the literature. On the other hand, HCP capture is driven by the values of K_{D} of the single HCP:peptide binding events, and since each HCP is present at very low concentration (≤1 nM), the binding affinity of the selected ligands is actually high, as observed in prior work on LigaGuard™ ligands.

### Example 4

Clearance of K. phaffii HCPs inflow-through mode: PichiaGuard vs. ion exchange resins. The purification of biopharmaceuticals expressed by K. phaffii relies almost ubiquitously on ion exchange (IEX) and mixed-mode (MM) chromatography. In particular, the post-capture steps of product polishing are being increasingly conducted in flow-through mode, which combines speed of operation with lower capital and operational costs. In this context, the IEX and MM resins employed in flow-through operations often include a quaternary amine (Q) moiety, which, as a strong cationic group, provides high binding capacity and strength of HCPs over a broad range of pH and conductivity values. Owing to their high capacity, IEX resins are often utilized at the capture step when affinity resins are not available. On the other hand, these resins lack binding selectivity and must be combined in a series of orthogonal chromatographic steps to ensure high product purity, which requires extensive process optimization and is often achieved at the expense of yield. The paradigm of flow-through affinity chromatography as a step dedicated to abating process-related impurities holds strong promise to overcome these challenges and offers an ideal route to de-risk platform processes for established products as well as products that do not benefit from a dedicated affinity resin. In prior work on mAb purification in flow-through mode, it was observed that LigaGuard™ resin outperform commercial IEX and MM resins on clarified and unconditioned CHO cell culture harvests, especially due to their lack of needing process optimization. In this study, an analogous comparison between PichiaGuard, LigaGuard™, and a commercial anion exchanger (AEX, Capto Q) and cationic MM (CaptoAdhere) resins was conducted by tracking HCP capture upon continuous loading of a clarified K. phaffii harvest.

To study the effect of pore size, the PichiaGuard was conjugated on two different polymethacrylate-based resins, one with pore diameter of 100 nm (Toyopearl 650M, TP-650M) and one with pore diameter of 5 nm (Toyopearl HW-50F, TP50F). Small proteins such as engineered antibody fragments, such as Fab or scFv, feature hydrodynamic radii ~2-4 nm [60,61] and are produced often using K. phaffii. Accordingly, the combination of size exclusion of the product and affinity capture of HCPs was explored as a means to improve product yield and purity under optimal conditions of linear velocity of loading. Accordingly, the flow-through process was operated at bioprocess-relevant values of residence time (RT, 1 minute) to develop an efficient flow-through process. Finally, similar to static binding tests, the harvest was conditioned in either phosphate buffer at pH 7.1 or acetate buffer at pH 5.7. The flow-through effluent was continuously collected and apportioned in fractions at regular intervals to evaluate the temporal profiles of protein binding—both as global HCP titers and single species via proteomics analysis.

The results presented in FIG. 2 provide a comparison of the HCP-clearing performance of the peptide-functionalized resin (PichiaGuard) vs. AEX, CHO LigaGuard™ and MM resins as a function of load in the two buffer systems. The PichiaGuard outperformed both LigaGuard™ and commercial resins when operated in acetate buffer at the RT of 1 min, providing a cumulative HCP LRV>2 (i.e., >100-fold reduction of HCPs) when loaded with up to 10 CVs, followed by LRVs of 1.5-2 (i.e., 30-100-fold reduction) for up to 20 CVs, 1.3-1.5 (i.e., 20-30-fold reduction) for up to 40 CVs, and plateauing at 1.3 for up to 100 CVs; LigaGuard™ resin afforded the second best HCP clearance profile, with LRVs of 1-1.5 for up to 10 CVs, 0.75-1 for up to 20 CVs, and plateaued to 0.45-0.55 CV when loaded up to 40 CVs. The reference AEX and MM resins provided a rather poor HCP clearance (without keen buffer or process optimization), consistently below a 10-fold reduction and plateauing to a LRV—0.5 (3-fold reduction) when loaded up to 40 CVs. A similar performance ranking was observed in phosphate buffer, with the sole exception of the AEX resin, which performed on par with the PichiaGuard resin across the entire loading range (40 CVs). The magnitude of HCP LRVs, however, decreased for all resins, thus mirroring the stating binding results discussed above: the highest drop was registered with PichiaGuard, whose HCP clearance activity in phosphate buffer was 10-fold lower than its acetate counterpart, while the performance of LigaGuard™ and CaptoAdhere was almost unaffected.

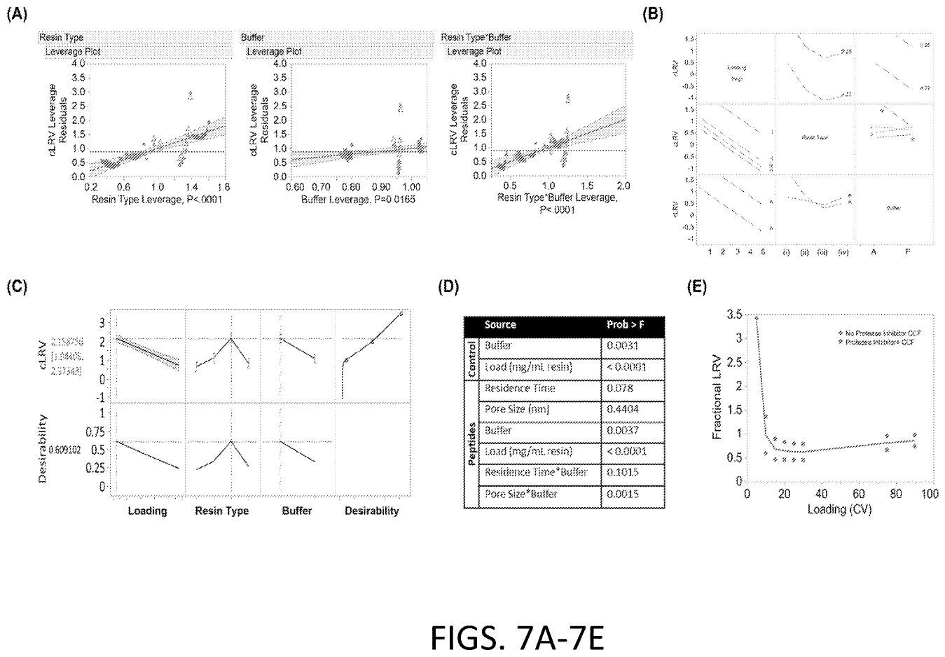

A number of conclusions can be drawn from these results: (i) peptide ligands significantly outperformed simple MM or IEX ligands, likely due to their broader range of non-covalent interactions and conformations; and (ii) fiber mat selection targeted to K. phaffii HCPs proved critical to identify an ensemble of bespoke ligands. While in fact the LigaGuard™ peptides delivered a higher HCP capture than MM or IEX moieties (Table 2), likely owed to the variety of interactions peptide ligands offer, the K. phaffii HCP-targeting peptides perform as custom affinity ligands—or at least, as more advanced than conventional mixed-mode ligands without the need for process optimization—by displaying both stronger binding strength and capacity and host selectivity. Furthermore, (iii) the PichiaGuard peptides ought to be utilized under conditions, such as the 20 mM acetate buffer at pH 5.7, that resemble the native physicochemical environment of K. phaffii harvests, namely acidic pH. Finally, (iv) around the 30^{th} CV of loading, it was observed the cumulative LRV plateau at ~1 and the corresponding fractional LRV become lower than 0.5 (FIG. 7E), indicating that the adsorbent has reached saturation. The 30^{th} CV mark corresponds to a load ratio of ~18 g of HCPs per L of resin, which corresponds to ~75% of Qₘₐₓ. This is a reasonable outcome given the relationship between dynamic vs. static binding and can be improved by adjusting the residence time as well as buffer composition and pH. Nonetheless, contrary to affinity resins that operate in bind-and-elute mode, the loading of ‘Guard’ resins is intended to reach halfway between the values of DBC_{10%} and Qₘₐₓ.

Table 2: Number of K. phaffi HR-HCPs captured by PichiaGuard, CHO LigaGuard™ and CaptoQ resins loaded with X-33 feedstocks (residence time: 1 minute) with HCP titer of ~0.4 mg/mL and conditioned in either 20 mM sodium acetate at pH 5.7- or 25-mM sodium phosphate at pH 7.2.

| | | | |
|---|---|---|---|
| | CHO | PichiaGuard- | PichiaGuard- |
| CaptoQ | LigaGuard ™ | TP50F | TP650M |

Acetate

| | | | | |
|---|---|---|---|---|
| Undetected | 19 | 18 | 19 | 18 |
| Not Captured | 15 | 19 | 5 | 10 |
| Captured | 10 | 11 | 20 | 16 |
| % | 40% | 42% | 80% | 62% |

Phosphate

| | | | | |
|---|---|---|---|---|
| Undetected | 0 | 8 | 0 | 0 |
| Not Captured | 29 | 20 | 17 | 8 |
| Captured | 15 | 15 | 27 | 36 |
| % | 34% | 41% | 61% | 82% |

Improving the clearance of HCPs from a given K. phaffii harvest using PichiaGuard resin can be easily achieved via process optimization. Conversely, achieving comparable purification under given conditions from fluids that differ substantially in the profile of physicochemical properties of their HCPs is far from a foregone conclusion. The global HCP removal was measured from two fluids—produced respectively with and without the addition of protease inhibitors to the cell culture medium—whose distribution of HCP molecular weights differed radically (FIGS. 2C-2D). To that end, a design-of-experiments (DOE) algorithm was implemented to explore systematically the effects of both categorical (i.e., buffer and resin) and continuous (i.e., buffer conductivity and pH, load ratio, and RT) variables to identify parameters that maximize HCP capture (FIGS. 7A-7D). The choice of resin, buffer, and interaction effects between these parameters were indeed found to be significant. The effect of RT appeared to be the most pronounced when using phosphate buffer: the highest HCP reduction (LRV ~3) was obtained at the RT of 1 min, which was therefore adopted for the remainder of this study. Notably, the combination of phosphate buffer and a smaller pore diameter of the resin led to higher HCP clearance (LR-~0.8-1) than its acetate counterpart (LRV~0.7-0.9), possibly due to effects of phosphate counterions on protein folding and increased ability to diffuse into smaller pores; however, limiting the total capture capacity. PichiaGuard-TP650M, however, afforded a superlative HCP reduction (LRV~3) and emerged as the resin of choice. As observed above, performing flow-through purification in acetate buffer consistently achieved a higher HCP clearance thus providing conclusive evidence for the adoption of acetate buffer for resin equilibration and, ideally, conditioning of the feedstock.

### Example 5

Tracking the removal of high-risk HCPs from K. phaffii cell culture harvests by PichiaGuard. In the established biopharmaceutical practice, the validation of a batch of therapeutic proteins consists in certifying that the residual HCP and hcDNA titers are below an acceptable limit for monoclonal antibodies (mAbs), these values are 100 ppm and 10 ppb, respectively. The measurement of residual HCP titer has been conducted for decades with ELISA kits. Like all assays that relies on polyclonal antibodies raised against multiple antigens, most ELISAs do not provide a full coverage of HCPs, but only of those that are abundant or more immunogenic to the animal host; furthermore, HCP aggregation and the formation of HCP:product complexes—a widespread phenomenon in bioprocess fluids—can impair assay readout. These elements, combined with the variability among assay lots and operator's performance, question the accuracy of HCP quantification provided by ELISA assays. Furthermore, in recent years, the growth of proteomics in biomanufacturing has shown that product batches with acceptable global level of impurities can contain amounts of single high-risk HCPs (HR-HCPs) that pose a serious threat to patient health (e.g., are toxic or immunogenic) or can degrade the mAb or its excipients during storage, resulting in reduced efficacy or harmful products. Recent studies have amply documented that commercial chromatographic resins may struggle to remove particular HR-HCPs: a number of these “persistent” HR-HCPs have been reported on both a process- and product-basis and have been linked to delays in clinical trials and process approval or the recall of drug batches.

Under this premise, the measurements of global residual HCPs presented above were complemented with tracking the individual HCPs captured by PichiaGuard via proteomics analysis. To this end, the effluents obtained in both acetate and phosphate buffers at the RT of 1 min were analyzed via LC-MS/MS by implementing the proteomics workflow outlined above for top-speed data dependent acquisition (DDA). Spectral interrogation using the Proteome Discoverer Suite was used to perform label-free quantification (LFQ) of HCPs in the flow-through fractions vs. the corresponding feedstocks (note: due to the lower number of proteins identified in K. phaffii harvests compared to HCP harvests, a ‘match between runs’ was implemented to preserve information from low-intensity peptide hits that that may have been masked by abundant contaminants like trypsin and any media components). Briefly, (i) data processing prior to analysis of variance (ANOVA) involved the exclusion of contaminants such as trypsin and keratin as well as the HCPs found in the effluents but not the load sample; (ii) the abundance of individual HCPs was used to calculate the removal values. It was noted that certain proteins were identified in some flow-through fractions to have an abundance ratio >1 (defined as the ratio of HCP abundance in the effluent sample vs. the load sample), suggesting that their presence in the load was masked by others in the load sample, more abundant peptides with similar sequence or charge which were not excluded.

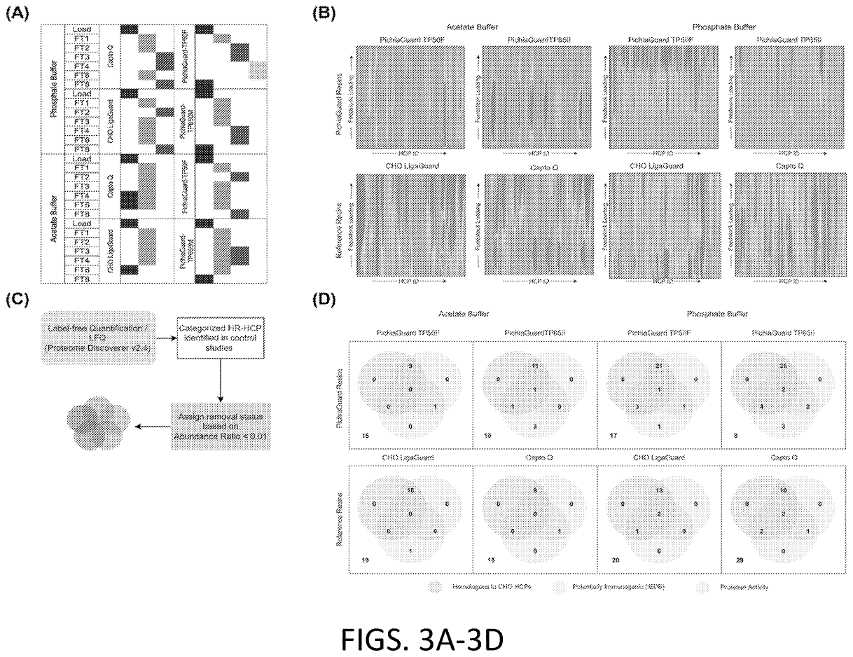

Three main indicators were adopted to evaluate the HCP-capture performance of different adsorbents, namely (a) global concentration and reduction; (b) between-group analysis, which presents the abundance trends of every HCP identified; and (c) clearance or persistence of high-risk HCPs (HR-HCPs). These indicators capture different variations within the study group and have been used collectively to make inferences. FIG. 3 presents the global landscape of the HCPs identified across all control experiments. The between-group (group=experiment of a particular resin with a particular buffer system) significance analysis, estimated using an iterative Tukey-Kramer HSD test known as the Newman-Kuels method between all pairs was used to construct a ‘connected-color’ plot, which connects fractions with a similar normalized mean abundance with the same color and ensues discontinuity with significant ‘within group’ difference (p-value >0.01). This test provided a picture of changes in overall abundance compared to the load and other fractions while other statistics and visualization techniques have been employed to track changes across individual species (data not shown but can be made available upon request).

To visualize and compare HCPs in successive flow-through fractions within and across buffer groups—(acetate, phosphate) and adsorbents (PichiaGuard-TP650M, PichiaGuard-TP50F resins, CHO LigaGuard™ and Capto Q resin)—FIG. 3B presents a reduced-dimension view of individual HCP abundance (Uniprot Accession numbers, left-to-right) vs. volume of effluent (flow-through fraction, bottom-to-top). Specifically, abundance ratios (fraction by load) are represented as contour plots wherein ratios ≥1 (in red) mark HCPs that were not cleared or whose presence was masked in the feedstock by other species but was detected in a flow-through fraction, whereas ratios ≤1 (veering from yellow to green as the value decreases) mark HCPs that were captured. At a glance, it was observed the ability of PichiaGuard resins to associate with most HCPs within both buffer systems compared to the reference resin counterparts. The contour plots provide visual indication of individual HCPs that were not captured throughout a significant part of the load due to insufficient HCP:ligand binding strength or possibly due to lack of charge/polarity of HCPs at the pH condition of the experiment, based on their isoelectric points.

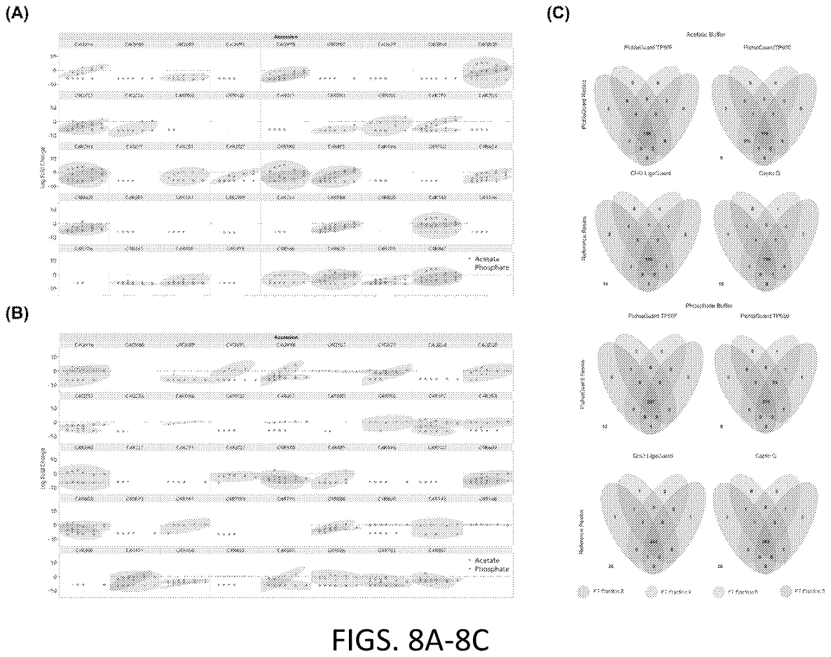

Notably, the proteomics analysis of the effluents enabled tracking of the capture of K. phaffii HR-HCPs by the various adsorbents (FIG. 3C), specifically focusing on proteins that are (i) homologous to known high-risk and persistent CHO HCPs; (ii) immunogenic, either reported or predicted based on their ability to generate MHC-II binding peptides; and (iii) possess proteolytic or other enzymatic activity, as described herein. The number of bound HR-HCPs across groups were classified according to their risk subtypes/bins (FIG. 3D). Corresponding logarithmic values of abundance variation of all identified HR-HCPs is presented in FIGS. 8A-8B, whereas the number of captured HR-HCPs as a function of loaded volume across all groups is reported in FIG. 8C.

From these results, the following key observations were drawn. Firstly, the PichiaGuard resins (FIG. 3B, bottom row) outperformed both LigaGuard™ and AEX (Capto Q) resins in retaining a higher number of K. phaffii HCPs, thus corroborating the results of total HCP quantification obtained from the total protein assays. It is noteworthy that this behavior is observed across all flow-through fractions for the HCPs, indicating a true lack of capture. As shown in FIG. 8C of the 187 HCPs identified in the acetate-conditioned feedstock, 180 were captured by PichiaGuard resins throughout the entire feedstock loading, while 3-5 were not captured; similarly, of the 318 HCPs in phosphate-conditioned feedstock (for a total of 335 unique K. phaffii HCPs identified), 302 were captured, while 8-12 were not captured. Secondly, PichiaGuard-TP650M and PichiaGuard-TP50F resin performed similarly in acetate buffer, whereas in phosphate buffer PichiaGuard-TP650M clearly outperformed its TP50F counterpart. The contour plots also show a greater extent of HCP dissociation from PichiaGuard resins in acetate media—especially from TP650M resins, likely a combined effect of pore size and residence time. Thirdly, PichiaGuard demonstrated the ability to clear up to 82% of HR-HCPs—including various aspartic proteases, ion protease homologs, etc.—whereas LigaGuard™ and CaptoQ resins only bound up to 42% of them. This corroborates the claim that ion-exchange resins, despite their satisfactory global HCP capture under optimized conditions, can fail to clear a number of HR-HCPs and are therefore unlikely to serve effectively as an HCP-scrubbing step prior to or in lieu of the affinity-based product capture step. Conversely, PichiaGuard makes an excellent tool for orthogonal HCP removal, thus safeguarding the performance and lifetime of affinity resins as well as promoting product quality and stability.

As is to be expected with any chromatographic adsorbent, process optimization in terms of loading ratio, flow rate, and buffer composition is to some extent needed. In the context of this study, however, these results aim to demonstrate the potential of PichiaGuard resin to increase the performance and robustness of current processes as well as provide a route towards platform processes with altogether novel design for isolating biopharmaceutics from Pichia harvests.

### Example 6

Purification of ScFv13R4 and mAb from K. phaffii harvests via flow-through affinity chromatography using PichiaGuard. Purification processes fully operated in flow-through mode are the epitome of continuous downstream biomanufacturing: their design minimizes process footprint and the number of tanks for storing service buffers, simplifies the pipework and control systems, and accelerates operations thus increasing productivity. Straight-through processes combining column chromatography and filtration unit operations have been proposed for the continuous manufacturing of mAbs from CHO cell harvests. These processes can be successfully designed based on the size of the target product, which is consistently larger than most of the process-related impurities. Conversely, small proteins are significantly more challenging products, as they remove filtration as an orthogonal method of separation and force reliance solely on chromatography. In this context, pseudo-continuous processes can be utilized by integrating periodic counter-current (PCC) or simulated moving bed (SMB) modes encompassing ion exchange chromatography, whose complexity increases both capital and operation costs. An effective alternative is offered by leveraging resins specifically tasked with HCPs and hcDNA clearance, like LigaGuard™ and PichiaGuard, which provide a product-agnostic route towards fully chromatographic straight-through processes for the purification of both mAb and non-mAb drugs.

Under this premise, having demonstrated the broad HCP-capture activity of PichiaGuard, experiments were conducted to evaluate its ability to purify therapeutic proteins from K. phaffii harvests in flow-through mode. To this end, an antibody fragment (ScFv13R4, MW~30 kDa and pI of 8.36) and a full antibody (referred to as ‘mAb’ hereon, MW~150 kDa and pI of 7.56) were adopted as model targets. Most importantly, to demonstrate the robustness of the flow-through affinity technology, the K. phaffii harvests were loaded on PichiaGuard post diafiltration into acetate buffer, until reaching a ratio >15 grams of HCPs per liter of resin (corresponding to DBC_{20%}). The analyses of the flow-through fractions—namely, global clearance of HCP and other impurities estimated and product yield—are reported in Table 3.

TABLE 3
Results of ScFv13R4 purification obtained by loading PichiaGuard-TP650 resin loaded
(residence time: 1 minute) with X-33 K. phaffi feedstock with ScFv13R4 titer of ~1 mg/mL,
HCP titer of ~0.4 mg/mL, and conditioned in 20 mM sodium acetate at pH 5.7.

| | Protein Loading | Ratio of product:non-product | Cumulative | Cumulative | Cumulative |
|---|---|---|---|---|---|
| Molecule | (g/L resin) | peaks (RP-HPLC) | HCP LRV | DNA LRV | ScFv Yield |
| ScFv13R4 | Load | 0.19 | — | | |
| | 2.10 | < LOD | 3.00 | 1.23 | 54.16% |
| | 3.90 | < LOD | 2.55 | | |
| | 5.10 | 2.06 | 1.92 | | |
| | 6.30 | 2.83 | 1.55 | | |
| | 7.50 | 1.77 | 1.34 | | |
| | 9.30 | 1.40 | 1.14 | | |
| | 14.40 | 1.09 | 1.01 | | |

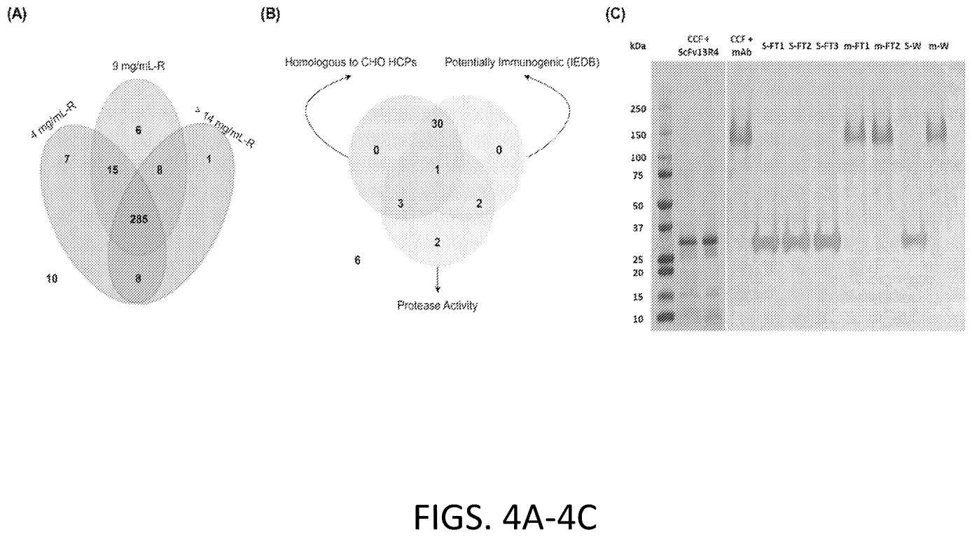

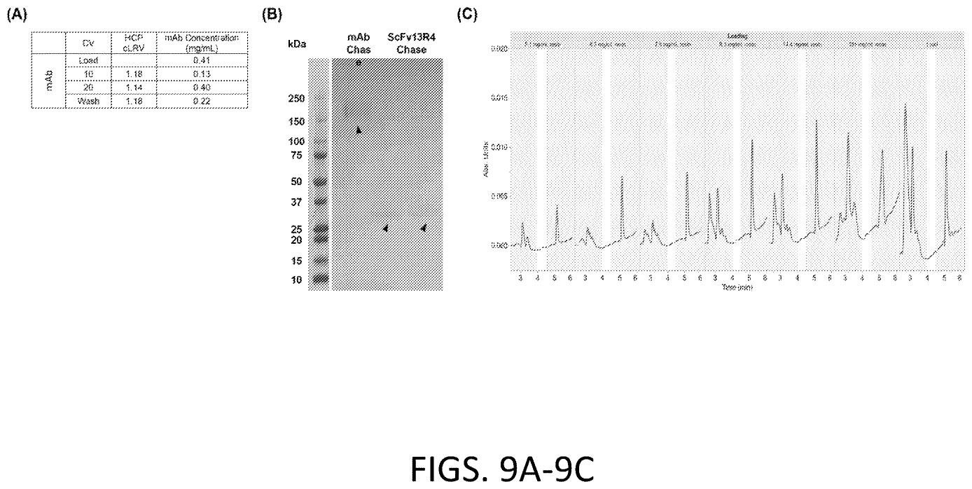

The purification of ScFv13R4 returned considerable values of impurity removal, with HCP clearance ranging from LRV>2.5 (~320-fold removal) to LRV>1.5 (~32-fold removal) when loading respectively up to 3.9 and 7.5 grams of HCPs per liter of resin, finally reaching LRV~1 at loading 14.4 mg/mL resin, at which a DNA LRV ~1.25 was also achieved. The electrophoretic analysis of the flow-through fractions in FIG. 4A provides at-a-glance presentation of impurity removal by PichiaGuard: particularly noteworthy is the comparison between the feedstock and effluents containing the ScFv13R4, which demonstrate the removal of contaminants both heavier and lighter than the product. Notably, the proteomics analysis of the effluents showed that, of the total 336 HCPs identified in the feedstock, the number of escaping species increased from 11 at the initial collection point of 4 g/L to merely 34 at the final pool, with 10 not being captured throughout. This corresponds to >84% of HCPs completely removed—chiefly among them, 38 out of 44 HR-HCPs—while the remainder ~15% HCPs were partially cleared. At the same time, the yield of ScFv13R4 reached only ~56% at the end of the loading phase (estimated using 660 nm BCA for 6×-His tagged ScFv13R4) but was recovered from the column by ‘chasing’ it with a high-conductivity buffer (FIG. 9B, chase not quantified). While the low titer of ScFv13R4 in the feed (~1 mg/L) made product detection challenging, the analysis of the flow-through fractions collected at a load value of >14 mg/mL resin demonstrated a level of HCP-HCP or HCP-product association (Table 3 and FIG. 9C); specifically, the reverse phase chromatograms show that the product-to-impurity ratio in the effluent increases as the loading progresses, translating in a growing product enrichment throughout the flow-through operation. The relationship between HCP-capture using PichiaGuard resins as a function of ScFv13R4 product concentration will be studied as the next step. Similar results were obtained from the purification of a full mAb (FIG. 9A): overall HCP clearance of 1.15 LRV was obtained up to a load of 15 mg/mL with a mAb product recovery of 80% estimated using an analytical Protein A HPLC assay.

Collectively, these results provide a strong proof-of-concept of PichiaGuard as a product-agnostic technology for impurity removal from K. phaffii cultures. At the same time, improvement in binding selectivity via optimizing ligand combinations or pretreatment of these cultures needs to be explored, along with the development of additional analytics.

### Example 7

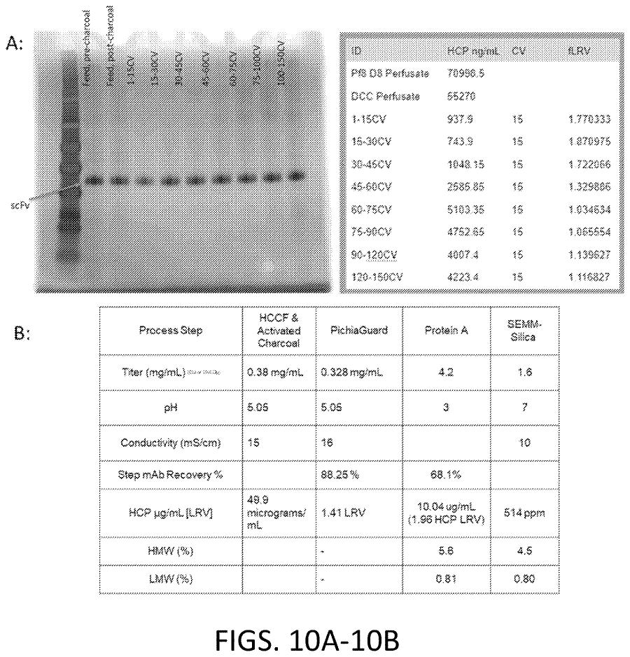

Experiments were conducted to characterize the performance of the peptide ligands of the present disclosure. FIG. 10 shows the successful integration of PichiaGuard with continuous fermentation processes that utilize Pichia pastoris to produce recombinant proteins. (A) Results of flow-through purification of an scFv fragment obtained using Silver stained SDS-PAGE (left) and Pichia pastoris Generation 2 HCP ELISA (right). Pigment interference from these cultures was first removed using dextran coated charcoal that was observed to nominally affect total HCP concentration within these samples (gel lane 3). 150 column volumes of this perfusate were loaded onto the PichiaGuard column where HCP removal of 1.7 logs was observed. (B) PichiaGuard was also utilized to purify continuously produced mAbs from Pichia fermentation where HCP reduction was demonstrated to 514 ppm after a 3-column purification process involving PichiaGuard and Protein A purification and no buffer optimization.

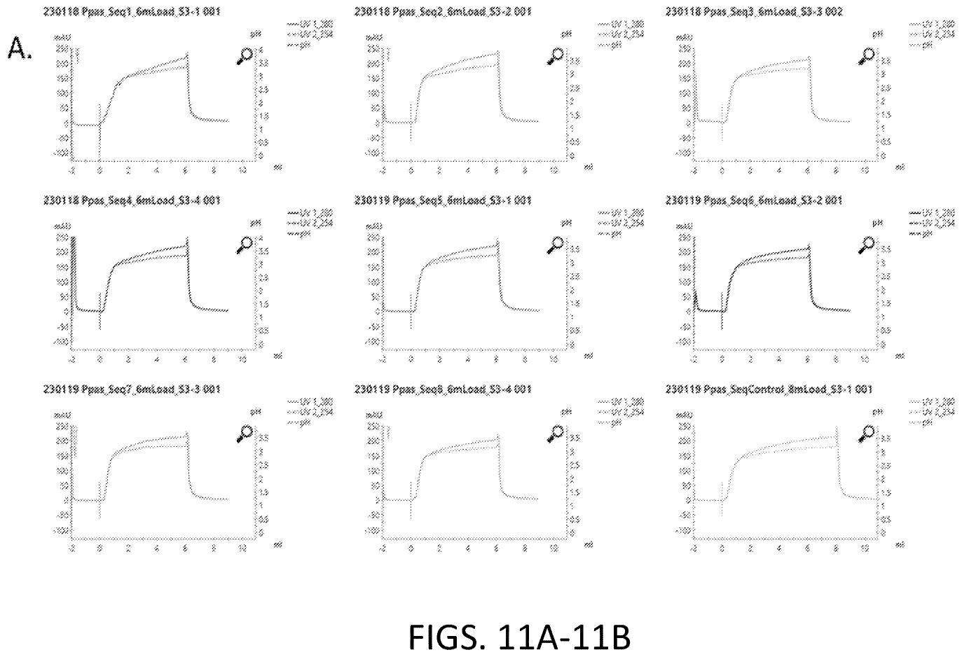

The initial breakthrough region of the chromatograms in FIG. 11A demonstrates qualitatively the uptake of HCP mass by each peptide, wherein the control PichiaGuard resin shows the slowest rate of saturation. FIG. 11B demonstrates HCP (target, left) and IgG (competitor, right) binding abilities of each peptide, evaluated as a function of total protein loading (contour). Within HCP binding, it was observed that sequences 5 through 8 display the highest capacity for HCPs, followed by 4, 3 and 1. The same sequences in order also show low to high IgG binding potential, generating a qualitative understanding of binding capacity (Y-axis) and binding selectivity (HCP/IgG). These results are later analyzed in conjunction with individual HCP binding information.

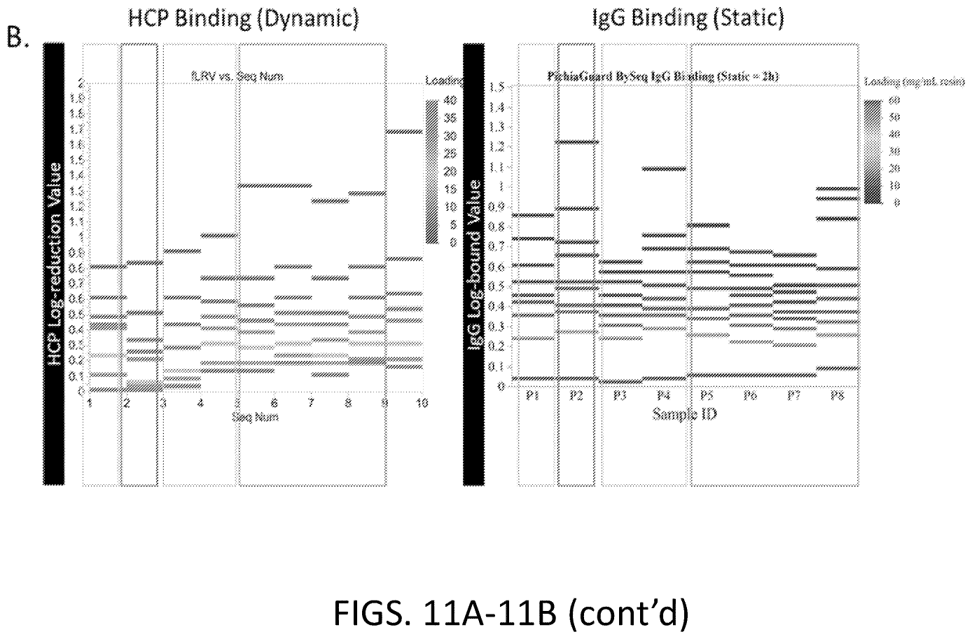

FIG. 12 summarizes the properties of HCPs derived from null Pichia pastoris fermentation utilized to perform peptide contribution characterization experiments. FIGS. 12A-12C compare HCP theoretical isoelectric point with charge, visualized based on HCP molecular weights, hydrophobicity and H-bonding abilities respectively, to identify apparent trends. While molecular weight and HCP charge were not correlated, a lower degree of hydrophobicity was observed with the most negatively charged HCPs—which also happened to mostly be H-bond acceptors. This suggests that these HCPs predominantly bound through electrostatic interactions. The most positive HCPs within the group (pI>10) displayed a high degree of hydrophobicity and H-bonding propensity. For the next set of analyses, HCPs were colored based on their abundance within the feed sample, indicated by their PSM (peptide spectrum matches) within label free quantification (FIG. 12D). While the largest proteins within the HCP repertoire were not the most abundant within the feed sample (FIG. 12E), most of these species displayed low propensities to engage in H-bonding, suggesting the adoption of alternate binding modes. A positive correlation was observed between HCP charge with H-bonding and hydrophobicity respectively (FIGS. 12F and 12G), indicating an increase in the potential to form H-bonds due to enhanced dipole potentials, with increasing positive charge on HCPs, and highlighted higher hydrophobic interactions due to the marginal magnitude of electrostatics.

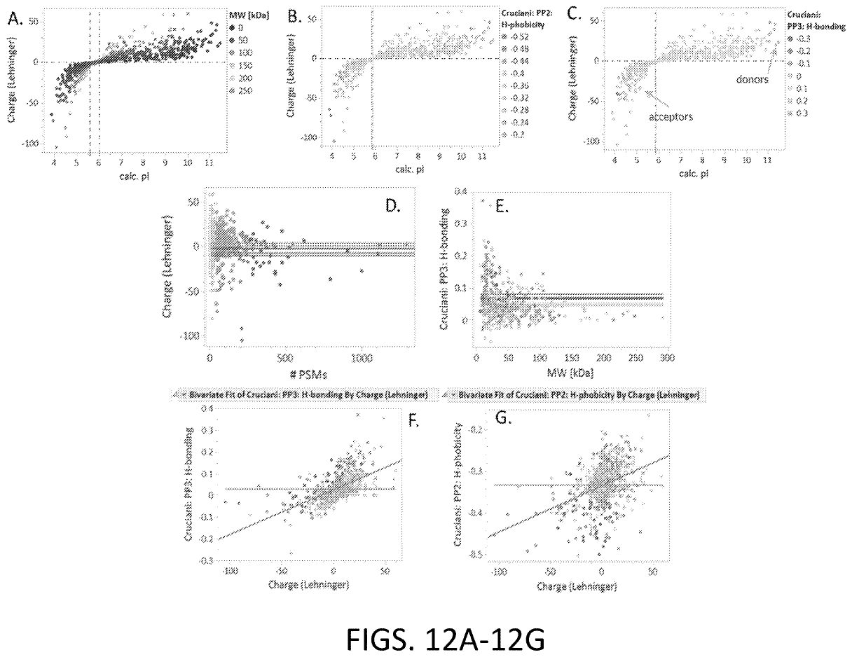

Individual HCP binding capabilities of each peptide are compared and shown in FIG. 13. The color spectrum diverging from blue to red indicates an increasing number of HCPs with specific molecular weight (Y-axis) and theoretical isoelectric points (X-axis) captured by each peptide. High HCP binding densities were observed with peptide 1 and 4, followed by 2, 3, 5, 7 and the least number of HCPs were bound by peptide 8. Peptides 1, 4, 5 and 7 displayed a broad coverage of HCPs with varying pI. However, a combination of the 8 peptides was observed to provide both maximum coverage and high HCP absolute HCP binding as seen in the left panel.

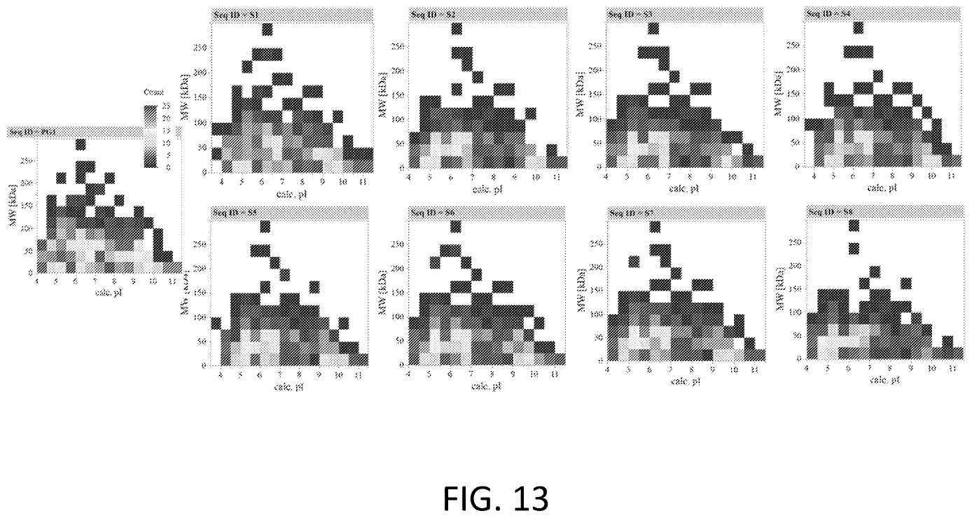

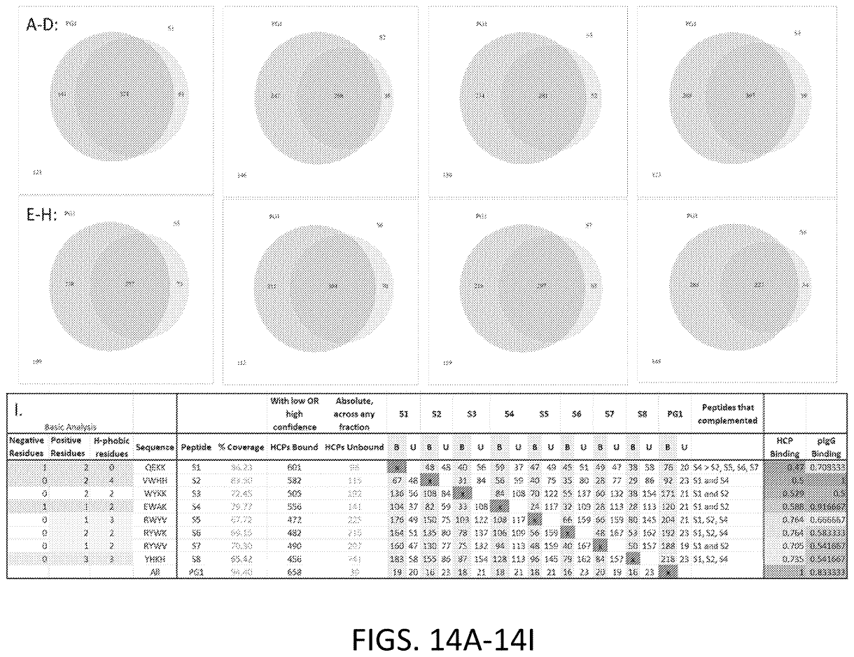

FIGS. 14A-14H compare the total number of identified HCPs bound by individual peptides with that of the PichiaGuard ensemble. The Venn diagrams indicate the proportional number or percentage of HCPs bound by the individual peptides in comparison to PichiaGuard, where the same trends were observed as in FIG. 12, with Peptides 1, 4, 6 and 7 binding most species, followed by peptides 3, 5, 2, and 8. In FIG. 14I, a comparison of peptide properties based on their composition reveals that the highest binders either consisted of a negatively charged or two positively charged amino acids. The remaining peptides were all found to have a substantial presence of hydrophobic residues, which was observed to lead to higher selectivity, with the exception of peptide 2, which displayed poor selectivity due to dense hydrophobicity. A combination of all these peptides as seen with the case of PichiaGuard hence achieves a balance between capacity and selectivity of HCPs within cell culture samples.

### 4. Materials and Methods

Materials. Fmoc-protected amino acids Fmoc-Gly-OH, Fmoc-Ser(tBu)-OH, Fmoc-Ile-OH, Fmoc-Ala-OH, Fmoc-Phe-OH, Fmoc-Tyr(tBu)-OH, Fmoc-Asp(OtBu)-OH, Fmoc-His(Trt)-OH, Fmoc-Arg(Pbf)-OH, Fmoc-Lys(Boc)-OH, Fmoc-Asn(Trt)-OH, Fmoc-Glu(OtBu)-OH, Fmoc-Pro-OH, Fmoc-Trp(Boc)-OH, Fmoc-Cys(Trt)-OH, and Fmoc-Leu-OH, the coupling agent azabenzotriazole titanium uronium hexafluorophosphate (HATU), diisopropylethylamine (DIPEA), piperidine, and trifluoroacetic acid (TFA) were sourced from ChemImpex International (Wood Dale, IL, USA). The Toyopearl AF-Amino-650M (PichiaGuard TP-650M) and HW-50F (PichiaGuard TP-50F) resins were obtained from Tosoh Bioscience (Tokyo, Japan). Phosphate buffered saline at pH 7.4, triisopropylsilane (TIPS), 1,2-ethanedithiol (EDT), anisole, Protease Inhibitor Cocktail, Kaiser test kits, and 3 kDa and 10 kDa MWCO centrifugal filters were from Millipore Sigma (St. Louis, MO, USA). N,N′-dimethylformamide (DMF), dichloromethane (DCM), methanol, N-methyl-2-pyrrolidone (NMP), sodium phosphate (monobasic), sodium phosphate (dibasic), hydrochloric acid, sodium acetate, glacial acetic acid, Formic acid, Tris-HCl at pH 7.5 and 8, Trypsin (100 μg), DTT, 660 nm Histidine-rich protein quantification and bicinchoninic acid (BCA) assay reagents were obtained from Fisher Chemicals (Hampton, NH, USA). Human IgG was obtained from Athens Bio (Atlanta, GA). Ammonium Bicarbonate was sourced from Acros, Sodium Deoxychlate, IAA and CaCl₂) from Sigma Aldrich and Zwittergent 3-16 was obtained from CalBioChem. Vici Jour PEEK chromatography columns (2.1 mm ID, 30 mm length, 0.1 mL volume) and polyethylene frits were obtained from VWR international (Radnor, PA, USA). The 10⁻²⁰% Tris-Glycine HCl SDS-PAGE gels, Urea and Coomassie blue stain were purchased from Bio-Rad Life Sciences (Carlsbad, CA, USA). Alexa Fluor 488 and 594 dyes, and Pierce dye removal columns were obtained from Thermo Fisher Scientific. HiPrep Desalting 26/10 column and Sepharose CM ion exchange resin were obtained from Cytiva.

Proteomics analysis. A label-free relative quantification workflow was developed to analyze the K. phaffii secretome using a bottom-up approach. Filter-aided sample preparation (FASP) was performed with 30 kDa MWCO filtration units, each passivated with 20 μL of 0.1M TRIS-HCl pH 8 prior to analyses. The starting volume for each sample was 200 μL (with varying protein amounts per sample) and the BCA assay was used to determine total protein amount in each sample volume. Addition of 15 μL of 50 mM dithiothreitol or DTT (in 0.1 M TRIS-HCl at pH 8) to each sample—with incubation for 30 minutes at 56° C.—served to cleave disulfide bonds in sample proteins. This was followed by further protein denaturation by the addition of 200 μL of 8 M urea (in 0.1 M TRIS-HCl at pH 8) to each sample, with vortex and spinning down. Each sample was next transferred onto a separate, passivated, 30 kDa MWCO filtration unit for FASP. The samples were then concentrated by centrifugation at 12,000 g for 15 minutes at 21° C. and the flow-through was discarded. An additional 200 μL of 8 M urea was added to each filtration unit, immediately followed by the addition of 64 μL of 200 mM iodoacetamide or IAA (in 8 M urea). Each MWCO filtration unit now carried 264 μL of solution (containing 50 mM IAA). Incubation for 60 minutes at room temperature in the dark ensured the alkylation of free sulfhydryl groups of cysteine in the sample proteins. This was followed by centrifugation at 12,000 g for 15 minutes at 21° C. Subsequent washing steps were performed, involving multiple rounds of centrifugation (12,000 g for 15 minutes at 21° C.) and resuspension into TRIS-based buffers (3 rounds with 2 M Urea with 10 mM CaCl₂ in 0.1 M TRIS-HCl at pH 8 and 3 rounds with the digestion buffer 0.1 M TRIS-HCl pH 7.5) before the addition of trypsin to the MWCO filters (1:50 enzyme/protein ratio—based on BCA assay results—in 0.1 M TRIS-HCl pH 7.5) for overnight proteolytic digestion. All flow-through solutions up to this point were discarded.

The overnight digestion was disrupted using a solution of 0.001% v/v Zwittergent with 1% v/v formic acid solution or quench buffer. This quench buffer was added in steps (with centrifugation at 12,000 g for 15 minutes at 21° C. in between steps, combining and collecting the flow-through solutions from a given sample) up to a total volume of 450 μL per sample. Each sample, now comprised of 450 μL of tryptic peptide solution, was lyophilized and stored at −20° C. until nanoLC-MS/MS. Upon reconstitution in 200 μL of Mobile Phase A (MPA: 98% v/v water, 1.9% v/v acetonitrile, 0.1% v/v formic acid), reverse-phase separation of the tryptic peptide fragments was carried out using a PepMap 100 C18 trap column (3 μm particle size, 75 μm ID, 20 mm length) in series with an EASY-Spray C18 analytical column (2 μm particle size, 75 μm ID, 250 mm length)—in a ‘trap-and-elute’ configuration—installed on an EASY nanoLC-1200 instrument (Thermo Scientific, San Jose, CA) interfaced with an Orbitrap-Exploris-480 (Thermo Scientific, Bremen, Germany). The flowrate was maintained at 300 nL/min. Peptides were eluted using a 105 minutes solvent gradient, ramping from 5% to 25% Mobile Phase B (MPB: 80% v/v acetonitrile, 19.9% v/v water, 0.1% formic acid) over 75 min, followed by another ramp to 40% MPB over 10 min, and then a steep ramp to 95% MPB in 1 min, at which point MPB was maintained at 95% for 17 minutes for column washing. Eluted tryptic peptides were ionized by subjecting them to 1.9 kV in the ion source for electrospray ionization. The ion transfer tube temperature was maintained at 275° C. The peptides were interrogated by full MS scan and data-dependent acquisition (DDA) MS/MS. DDA was performed as a 105-minute nanoLC-MS/MS method. Scan parameters for full MS were: 120,000 resolution, RF Lens of 40%, maximum injection time of 120 ms, scan range of 375-1,600 m/z, with normalized AGC setting at 300%. Dynamic exclusion (with 20 s exclusion duration) was used to minimize re-interrogation of pre-sampled precursors. Cycle time was set at 1.5 s. Charge states 2-6 were included in the analysis. An intensity threshold of 1.5 10⁴ was set for ddMS². Scan parameters for ddMS² were: 15,000 resolution, inclusion window of 1.5 m/z, maximum injection time of 21 ms, HCD collision energy of 30% (fixed), with normalized AGC setting at 100%. Quality control in nanoLC-MS/MS analyses involved blank runs, as well as standard BSA digest and standard HeLa digest runs at regular intervals within the length of the sample queue.

Post-acquisition data analysis and automated LFQ was performed by Proteome Discoverer v. 2.4 (PD, Thermo Scientific, San Jose, CA) with appropriate grouping variables. The raw nanoLC-MS/MS files were interrogated against the Pichia pastoris/Komagataella phaffii protein database (4,983 sequences) downloaded from UniProt. The data was also searched against a contaminants database (69 sequences) to identify potential contaminants during the experiments. These databases were searched with the following parameters: trypsin (full) as the digesting enzyme, a maximum of 3 missed trypsin cleavage sites allowed, 5 ppm precursor mass tolerance, 0.02 Da fragment mass tolerance, dynamic modifications on methionine (oxidation), N-terminus (acetyl), methionine (met-loss+acetyl), methionine (met-loss), as well as static carbamidomethyl modifications on cysteine residues. The SEQUEST HT algorithm was employed in data interrogation. This algorithm compares the observed peptide MS/MS spectra and theoretically derived spectra from the target database(s) to assign appropriate quality scores. These scores and other important predictors are combined in the algorithm, which assigns a composite score to each peptide. The overall confidence in protein identification is increased with increasing number of distinct amino acid sequences identified. Therefore, proteins are normally categorized into a different priority group depending on whether they have only one or multiple unique tryptic peptide sequences of the required peptide identification confidence. Percolator peptide validation was based on the q-value (adjusted p-value) and minimal false discovery rate (FDR)<0.01 was selected as a condition for successful tryptic peptide assignments.

Design of K. phaffii strain X-33 producing ScFv13R4. The DNA sequence encoding the ScFv13R4 protein was first codon-optimized for efficient expression in Pichia pastoris wild type X-33 (Life Technologies Ltd, Grand Island, NY). The gene was amplified via PCR using primers scFv13R4 aF1 (GCATGAATTCATGGCAGAAGTCCAATTAG) and scFv13R4 αR1 (GAATGCGGCCGCTCATAAGACTGCCAACTTAGTACC) carrying the extra restriction sites EcoRI and NotI (New England Biolabs, Beverly, MA), respectively. The PCR product was cloned in the vector pPICZα-A (Life Technologies Ltd, Grand Island, NY) at EcoRI and NotI sites generating a fusion protein ScFv13R4 to both His and C-myc tags present on the expression vector. After electroporation in E. coli Stb14 and selection on LB agar plates supplemented with zeocin at g/mL, positive recombinant plasmids were identified using colony PCR. One recombinant plasmid was linearized with the restriction enzyme SacI and introduced into Pichia pastoris X-33 via electroporation. Selection was performed on YPD plates supplemented with zeocin at a concentration of 1000 g/mL and positive clones were screened with colony PCR.

Production of K. phaffii WT (null-X-33) and ScFv13R4 cultures. Overnight culture was initiated from a single colony in 3 mL of YPD medium and incubated at 30° C. under agitation at 350 rpm. A volume of 1 mL of this culture was used as the inoculum to initiate a new 50 mL culture in YPD medium, which was grown at the same conditions for 24 hours. In order to adapt the cells to the production medium, cells were centrifuged at 3000 g for 10 minutes and resuspended in 50 mL of salt media BFM21 containing glycerol at 40 g/L (3.5 mL of 85% H₃PO₄, 0.119 g CaSO₄, 2.4 g K₂SO₄, 1.950 g MgSO₄:7H₂O, 0.650 g KOH, 0.940 g sodium citrate, 5.0 g ammonium sulfate, 1.537 g NaOH, 31.75 mL glycerol, 40 mL 1.0 M acetate buffer, 0.25 mL antifoam 240, pH 5.0) and supplemented with trace elements PTM4 (12 ml/L: 1275 μL of H2SO₄, 0.5 g CuSO₄:5H₂O, 0.020 g NaI, 0.750 g MnSO₄, 0.05 g Na₂MoO₄:2H₂O, 0.005 g H₃BO₃, 0.125 g CoCl₂, 1.675 g ZnCl₂, 5.4 g FeSO₄, 0.050 g added per 250 mL of medium). Cell growth was allowed to proceed for 24 hours at 30° C. under agitation at 350 rpm. This culture was in turn used to inoculate a 1-L working volume bioreactor containing BFM21 supplemented with PTM4 (12 ml/L) medium and glycerol (40 g/L) starting at low optical density (OD_{550 nm}~2). Fermentation was conducted in multiple bioreactors using the following parameters: pH controlled at 5 using 28% v/v ammonium hydroxide and 80% v/v phosphoric acid; temperature at 30° C.; and dissolved oxygen maintained at 30% in cascade with agitation. The initial glycerol was exhausted within 24 hours of fermentation, after which a glycerol feed phase was initiated, and the fermentation was continued until the fluid reached an OD_{550 nm}~100. At this point, the suspension from one reactor was collected and centrifuged at 4000 g for 30 minutes to remove the wet cell mass, while ensuring minimal cellular rupture and intracellular protein release. Simultaneously, a methanol feed phase was started in the other bioreactor by adding it at a concentration of 0.10% v/v at 0.3 mL/min while maintaining the dissolved oxygen at 30%, to induce ScFv13R4 production. After 48 hours of fermentation in methanol and upon achieving a cell density of OD_{550 nm}~300, the cells were harvested via centrifugation at 3000 g for 10 minutes. The supernatant was then filtered through a 0.45 μm filter before further use.

Peptide synthesis. A tetrameric one-bed one-peptide library in the format X₁-X₂-X₃-X₄-GSG was prepared on HMBA-ChemMatrix resin via direct solid-phase Fmoc/tBu peptide synthesis using a Syro I automated peptide synthesizer (Biotage, Uppsala, Sweden), as described in prior work. Amination of Toyopearl HW-50F resin was performed by incubating 1 g of resin with 2 mmol of carbonyldiimidazole in 30 mL of acetone for 30 minutes at 25° C. under mild agitation, followed by rinsing in acetone and incubation with 2 mmol of tris(2-aminoethyl) amine under the same conditions, to obtain an amine density of ~0.2 mmol per gram of resin. The peptide sequences were synthesized on Toyopearl AF-Amino-650M (TP650M) and aminated HW-50F resins (TP50F) following the same protocol using an Alstra automated peptide synthesizer (Biotage, Uppsala, Sweden). Upon completing chain elongation, the peptides were deprotected using TIPS deprotection cocktail (95% v/v TFA, 2.5% v/v TIPS, 2.5% v/v water). The PichiaGuard-TP650M and PichiaGuard-TP50F adsorbents were formulated by combining equal volumes of the above-listed peptide-functionalized resins.

Protein labeling and formulation of a screening mix. Human IgG and the HCPs contained in the P. pastoris (X-33 strain) cell culture fluid and were labeled using NHS ester AlexaFluor dyes AF488 (green) and AF594 (red), respectively. Specifically, each dye was initially dissolved in dry DMSO at the concentration of 10 mg/mL. Following diafiltration of the X-33 cell culture fluid into PBS at pH 7.4 using 3 kDa MWCO filters, 100 μL of K. phaffii HCPs at the concentration of 0.5 mg/mL were incubated with 1 μL of AF594; in parallel, 100 μL of a solution of IgG at 5 mg/mL in PBS at pH 7.4 was incubated with 1 μL of AF594. The labeling reactions were allowed to proceed for 3 hours at 25° C. under mild agitation. Spin columns were used to remove the unreacted dyes. The labeled proteins were then mixed to obtain a screening mix comprising AF488-IgG at 2 mg/mL and AF594-HCPs at 0.2 mg/mL. A volume of 20 microliters of OBOP library beads were then incubated with 1 mL of screening mix overnight at 4° C. under mild agitation and washed with PBS before proceeding with the screening protocol.

Library Screening. The OBOP tetrameric library was screened against the screening mix using a microfluidic device previously developed. Briefly, the microfluidic device comprises an imaging chamber which hosts individual library beads in rapid sequence and is installed in a DMi8 microscope (Leica, Wetzlar, Germany) equipped with an ORCA-Flash4.0 V3 Digital CMOS camera with W View Gemini image splitting optics (Hamamatsu, Shizuoka, Japan). A MATLAB® GUI developed to operate this screening device was implemented to visualize the library beads under the green (human IgG; excitation/emission: 488/496 nm) and red (K. phaffii HCPs: excitation/emission: 594/617 nm) channels. Library beads with green-only or green-and-red fluorescent emission were discarded, whereas beads with strong red-only fluorescent emission were collected as positive leads and analyzed via Edman Degradation using a PPSQ 33-A Protein Sequencer (Shimadzu, Kyoto, Japan) to identify the peptide sequences carried thereon.

Diafiltration of feedstocks. Prior to chromatographic purification, the clarified X-33 cell culture harvests were diafiltered to remove small media component, chiefly glycerol, and unreacted protease inhibitors as well as adjust the conductivity and pH using a HiPrep™ 26/10 column (Cytiva, Marlborough, MA) into two different buffers, namely 20 mM sodium acetate at pH 5.7- and 25-mM sodium phosphate at pH 7.2. The HCP titers were varied respectively between 0.3 mg/mL and 0.5 mg/mL in both the acetate-conditioned and phosphate-conditioned feedstocks as estimated by BCA.

Static and dynamic HCP binding capacity measurements. Static binding experiments were conducted by incubating aliquots of 30 μL of settled PichiaGuard-TP650M or PichiaGuard-TP50F resin in a 12-well plate (WP) with acetate-conditioned and phosphate-conditioned X-33 feedstocks featuring HCP titers of 0-1 mg/mL for 3.5 hours at 25° C. under mild agitation (300 rpm). The plate was then spun down at 3000 rpm for 10 minutes and the supernatants were collected and analyzed to determine the residual HCP titer via BCA. Finally, the values of static binding capacity were calculated via mass balance. The dynamic binding capacity was measured via breakthrough experiments (DBC_{20%}) by loading ~6 mL of acetate-conditioned or phosphate-conditioned X-33 feedstocks featuring a HCP titer of ~0.4 mg/mL onto 0.1 mL column packed with chromatographic resin—namely, PichiaGuard-TP650M and PichiaGuard-TP50F resin, Capto Q or CaptoAdhere (Cytiva), or CHO LigaGuard™ (LigaTrap Technologies, Raleigh, NC)—pre-equilibrated with the respective buffers—namely, 20 mM sodium acetate at pH 5.7 and 25 mM sodium phosphate at pH 7.2—at a residence time (RT) of 1 minute until saturation was reached. The effluent was continuously monitored via spectrophotometry at 280 nm and apportioned in 0.5 mL fractions that were finally analyzed to determine the residual HCP titer via BCA.

Purification of ScFv and mAb via flow-through affinity chromatography using PichiaGuard. PichiaGuard resin was initially packed in a 0.1 mL column and equilibrated with 20 mM sodium acetate at pH 5.7 at 0.2 mL/min for 15 minutes. Two K. phaffi harvests were utilized as feedstock in this study—the first one featuring a ScFv titer of ~1 mg/L and an HCP titer of ~0.6 mg/mL, while the second one featuring a mAb titer of ~0.4 mg/mL and an HCP titer of ~0.5 mg/mL—both conditioned in 20 mM sodium acetate at pH 5.7. A volume of 4 mL of harvest was then loaded on the column at the residence time (RT) of 1 minute and flow-through fractions of 0.3 mL were collected throughout the load and final column wash for analytical characterization. All purification studies were performed using an AKTA pure (Cytiva, Chicago, IL, USA) while monitoring the effluents using UV spectroscopy at 280 nm.

Reverse phase (C18 RP-HPLC) analysis for the quantification of ScFv13R4 titer and purity. The titer of ScFv13R4 in the collected chromatographic fractions was measured via C18 RP-HPLC using a Zorbax C18-SB column (Agilent, Santa Clara, CA) installed on a Waters Alliance 2690 HPLC. The column was equilibrated by flowing 95% mobile phase B (MPB: 0.5% v/v TFA in acetonitrile) and 5% mobile phase A (MPA: 0.5% v/v TFA in water), at a flowrate of 0.5 mL/min. A 20-minutes gradient method (95-45% MPB) was conducted at the constant flow rate of 0.5 mL/min to achieve separation. Pure ScFv13R4 expressed in E. coli cells was utilized to identify the product retention time and calculate the ScFv13R4 purity in the chromatographic fractions.

Other Analytical Assays. Total protein and P. pastoris HCP titers in control studies were measured via BCA assay (Pierce BCA by ThermoFisher, NC) and Pichia pastoris HCP ELISA kit (Generation 2, Cygnus Technologies, SC) following the manufacturer's protocol. Product purity was evaluated using 660 nm BCA for histidine rich proteins and SDS-PAGE analysis under non-reducing conditions. mAb was quantified using an analytical proteinA method as described previously.

Analysis of PichiaGuard Peptide Contributions to HCP Binding: Contributions of individual peptides comprising PichiaGuard were discerned in terms of their binding to Pichia pastoris HCPs “by-sequence”. These studies comprised of the flow-through purification of null Pichia culture fluids (no recombinant protein), subsequently purified using chromatographic columns packed with individual peptides. The fluids were purified at a 1-minute residence time and flow-through fractions were collected across time for the identification of bound and unbound HCPs (see chromatograms in FIG. 11A). These fractions were analyzed using total protein quantification assay (BCA) and compared with that of the load sample to compute HCP log-reduction value for each individual sequence (FIG. 11B, left). Simultaneously, 25 μL of each peptide was incubated with a range of IgG concentrations (0.1-30 mg/mL resin) overnight to understand their binding propensities to the competitor molecules under non-competitive conditions. Protein content in the supernatant was estimated and compared to the initial concentrations to observe IgG binding (FIG. 11B, right). All flow-through fractions were analyzed through the proteomics workflow (as described further herein) to identify unbound HCPs and overall, correlate peptide and HCP properties. Briefly, label free quantification was performed to identify HCPs within each flow-through fraction and compared to the feed sample used within the experiment. The data was first grouped as a function of time for each peptide and data filters were applied based on the abundance ratio to label bound and unbound HCPs, at different confidence levels based on the number of fractions they were identified in. Concurrently, the “Peptides” package within RStudio was utilized to calculate sequence-based properties of each HCP such as polarity, H-bonding, hydrophobicity and charge. A collection of this data was analyzed to derive contextualized understanding of HCP binding per individual peptide with respect to the peptide properties.

What is claimed is:

1. A composition for purifying a target biologic from a biological fluid, wherein the composition comprises at least one peptide ligand that is at least four amino acids in length and comprises: (i) at least one charged amino acid, and (ii) at least one amino acid comprising a side chain capable of hydrogen bond formation.

2. The composition of claim 1, wherein the at least one charged amino acid is arginine (R), histidine (H), lysine (K), and/or glutamate (E).

3. The composition of claim 1 or claim 2, wherein the at least one charged amino acid is not aspartate (D).

4. The composition of any one of claims 1 to 3, wherein the at least one amino acid capable of hydrogen bond formation is tyrosine (Y), tryptophan (W), and/or glutamine (Q).

5. The composition of claim 4, wherein the at least one amino acid capable of hydrogen bond formation is not phenylalanine (F).

6. The composition of any one of claims 1 to 5, wherein the at least one peptide ligand comprises alanine (A) or valine (V).

7. The composition of any one of claims 1 to 6, wherein the at least one peptide ligand does not comprise isoleucine (I), leucine (L), or proline (P).

8. The composition of any one of claims 1 to 7, wherein an arginine (R) residue is flanked by a tyrosine (Y) residue and/or a tryptophan (W) residue.

9. The composition of any one of claims 1 to 8, wherein a valine (V) residue is flanked by a tyrosine (Y) residue and/or a tryptophan (W) residue.

10. The composition of any one of claims 1 to 9, wherein an alanine (A) residue is flanked by a lysine (K) residue.

11. The composition of any one of claims 1 to 10, wherein the at least four amino acids are hydrophilic amino acids.

12. The composition of any one of claims 1 to 10, wherein at least two of the at least four amino acids are hydrophobic amino acids.

13. The composition of any one of claims 1 to 12, wherein the at least one peptide ligand is selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

14. The composition of any one of claims 1 to 12, wherein the at least one peptide ligand comprises at least two peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

15. The composition of any one of claims 1 to 12, wherein the at least one peptide ligand comprises at least three peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

16. The composition of any one of claims 1 to 12, wherein the at least one peptide ligand comprises at least four peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

17. The composition of any one of claims 1 to 12, wherein the at least one peptide ligand comprises at least five peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

18. The composition of any one of claims 1 to 12, wherein the at least one peptide ligand comprises at least six peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

19. The composition of any one of claims 1 to 12, wherein the at least one peptide ligand comprises at least seven peptide ligands selected from the group consisting of: YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

20. The composition of any one of claims 1 to 12, wherein the composition comprises YHKH (SEQ ID NO: 1), RYWK (SEQ ID NO: 2), RWYQ (SEQ ID NO: 3), EWAK (SEQ ID NO: 4), and QEKK (SEQ ID NO: 5), WYKK (SEQ ID NO: 6), VWHH (SEQ ID NO: 7), and RYWV (SEQ ID NO: 8), or any derivatives or variants thereof.

21. The composition of any one of claims 1 to 20, wherein the at least one peptide ligand binds at least one host cell protein (HCP), at least one high-risk HCP, at least one host cell nucleic acid, aggregates of the target biologic, and/or an impurity derived from the target biologic.

22. The composition of claim 21, wherein the at least one peptide ligand exhibits a K_{D}<10⁻⁵ M for the HCP, the host cell nucleic acid, the aggregates of the target biologic, and/or the impurity derived from the target biologic.

23. The composition of any one of claims 1 to 22, wherein the at least one peptide ligand comprises a linker.

24. The composition of claim 23, wherein the linker is bound to the C-terminus of the peptide ligand, and wherein the linker comprises a Glyₙ or a [Gly-Ser-Gly]ₘ, wherein 6≥n≥1 and 3≥m≥1.

25. The composition of any one of claims 1 to 24, wherein the at least one peptide ligand is bound to a solid support.

26. The composition of claim 25, wherein the solid support comprises a non-porous or porous particle, a membrane, a plastic surface, a fiber or a woven or non-woven fiber mat, a hydrogel, a microplate, and/or a microfluidic device.

27. The composition of claim 25 or claim 26, wherein the solid support comprises polymethacrylate, polyolefin, polystyrene, polyester, polyether, polysaccharide, iron oxide, silica, titania, zirconia, and/or derivatives thereof.

28. The composition of any one of claims 1 to 27, wherein the biological fluid comprises a cell culture supernatant and/or a cellular lysate.

29. The composition of claim 28, wherein the biological fluid is derived from a yeast cell.

30. The composition of claim 29, wherein the yeast cell is selected from the group consisting of P. pastoris, S. cerevisiae, and S. boulardii, or any derivatives or variants thereof.

31. The composition of any of claims 1 to 30, wherein the target biologic is one or more of a protein, peptide or polypeptide; an oligonucleotide or a polynucleotide; a virus or a virus-like particle; an exosome or an extracellular vesicle; a cell or cell organelle; or a small molecule.

32. The composition of any of claims 1 to 26, wherein the biological fluid comprises a pH from about 3.0 to about 9.0.

33. The composition of any of claims 1 to 27, wherein the biological fluid comprises a conductivity of about 1 mS/cm to about 50 mS/cm.

34. The composition of any of claims 1 to 33, wherein the at least one peptide ligand exhibits a maximum equilibrium binding capacity (Qₘₐₓ) of at least about 5.0 g of host cell proteins per liter of resin.

35. The composition of any of claims 1 to 33, wherein the at least one peptide ligand exhibits a dynamic binding capacity (DBC_{10%}) from about 5 g/L resin to about 70 g/L resin.

36. An adsorbent comprising any of the peptide ligands of claims 1 to 35.

37. A method of purifying a target biologic from a biological fluid, the method comprising:
contacting the composition comprising the at least one peptide ligand of any of claims 1 to 35, or the adsorbent of claim 36, with a biological fluid comprising a target biologic; and
collecting an effluent in flow-through mode, wherein the effluent comprises the target biologic;
wherein the at least one peptide ligand binds and retains at least one host cell protein (HCP), at least one high-risk HCP, at least one host cell nucleic acid, aggregates of the target biologic, and/or an impurity derived from the target biologic.

38. The method of claim 37, wherein the method further comprises performing affinity chromatography on the biological fluid comprising the target biologic before or after contacting the composition comprising the at least one peptide ligand of any of claims 1 to 35.

39. The method of claim 37 or claim 38, wherein the method is performed under static binding conditions.

40. The method of claim 37 or claim 38, wherein the method is performed under dynamic binding conditions.

41. The method of any one of claims 37 to 39, wherein the method results in a yield of the target biologic of at least about 50%.

42. The method of any one of claims 37 to 39, wherein the method results in a purity of the target biologic of at least about 75%.


SEQUENCE LISTING (8 sequences)

SEQ ID NO: 1 - protein, 4 aa, Synthetic construct
YHKH

SEQ ID NO: 2 - protein, 4 aa, Synthetic construct
RYWK

SEQ ID NO: 3 - protein, 4 aa, Synthetic construct
RWYQ

SEQ ID NO: 4 - protein, 4 aa, Synthetic construct
EWAK

SEQ ID NO: 5 - protein, 4 aa, Synthetic construct
QEKK

SEQ ID NO: 6 - protein, 4 aa, Synthetic construct
WYKK

SEQ ID NO: 7 - protein, 4 aa, Synthetic construct
VWHH

SEQ ID NO: 8 - protein, 4 aa, Synthetic construct
RYWV